(19) United States
(12) Patent Application Publication
(10) Pub. No.: US 2026/0234101 A1
(43) Pub. Date: Aug. 13, 2026

(54) COMPOUNDS, THEIR COMPOSITION AND THEIR USE AS NEMATICIDES

(71) Applicants: Komal BHUKHANWALA, Mumbai (IN); Venkata Siva Prasada Rao LINGAM, Navi Mumbai (IN)

(72) Inventors: Komal BHUKHANWALA, Mumbai (IN); Venkata Siva Prasada Rao LINGAM, Navi Mumbai (IN)

(21) Appl. No.: 19/153,911

(22) PCT Filed: Feb. 19, 2024
(86) PCT No.: PCT/IN2024/050177

§ 371 Date: Aug. 5, 2025

(30) Foreign Application Priority Data
Feb. 17, 2023 (IN) 202321011330

(51) Int. Cl. C07C 279/14; A01N 47/44; A01P 5/00; C07D 209/14; C07D 213/53; C07D 307/52; C07D 317/58; C07D 333/22

(52) U.S. Cl. CPC C07C 279/14; A01N 47/44; A01P 5/00; C07D 209/14; C07D 213/53; C07D 307/52; C07D 317/58; C07D 333/22

(57) ABSTRACT

The present invention provides a compound of formula (I)

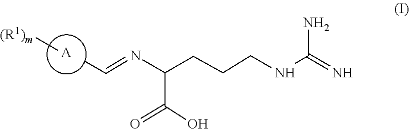

or agrochemically acceptable salt, stereoisomer, enantiomer, tautomer, or N-oxide thereof and their agriculture composition and their use for treating or controlling the parasitic nematode or the nematode infection in agriculture crops.

### FIELD OF THE INVENTION

The invention relates to compounds having nematocidal activity, their agricultural composition and use of such compounds for treating or controlling the parasitic nematode or nematode infection in agriculture crops.

### BACKGROUND OF THE INVENTION

Nematodes are active, flexible, elongate, organisms that live on moist surfaces or in liquid environments, including films of water within soil and moist tissues within other organisms. Many species of nematodes have evolved to be very successful parasites of plants and animals and are responsible for significant economic losses in agriculture. Nematodes are known to affect the yield, growth, and health of crops and plants. The physiological changes in the host plant's roots caused by larvae and/or adult nematodes can lead to the formation of galls, which causes a disruption of the vascular system of the plant's roots. Root elongation can stop completely and inadequate supply of water and nutrients provided by the reduced root system can result, causing foliage chlorosis and/or wilt, as well as stunting of growth, any of which can result in low yield or death. In addition, nematodes can cause physiological effects leading to an increase in the susceptibility of plant roots to bacteria and/or fungi attack, including bacteria and/or fungi the plant would otherwise resist. Such attack can lead to extensive secondary decay and rotting. Hence, the control of plant parasitic nematodes is extremely important in achieving high crop efficiency.

Due to widespread development of resistance to anthelmintic agents in nematode parasites, nematodes continue to cause problems despite the available therapeutic agents. The need continues for new compounds which are more effective, less costly, less toxic, environmentally safer or have different modes of action.

### SUMMARY OF THE INVENTION

The present invention provides a compound of formula (I),

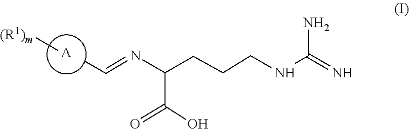

or agrochemically acceptable salt, stereoisomer, enantiomer, tautomer, or N-oxide thereof,
wherein, ring A, R¹ and ‘m’ are as defined in detailed description. The present invention further relates to an agriculture composition of compound of Formula (I) and their use for treating or controlling the parasitic nematode or the nematode infection in agriculture crops.

### DESCRIPTION OF THE INVENTION

### Definitions

In describing the embodiment of the invention, specific terminology is chosen for the sake of clarity. However, it is not intended that the invention be limited to the specific terms so selected and it is to be understood that such specific terms include all technical equivalents that operate in a similar manner to accomplish a similar purpose. It is understood that any numerical range recited herein is intended to include all subranges subsumed. Also, unless denoted otherwise percentage of components in a composition are presented as weight percent.

The terms “a” or “an”, as used herein, are defined as one or more than one. The terms “including” and/or “having”, as used herein, are defined as comprising (i.e., open language).

The term “salt” includes salts prepared from bases or acids including inorganic or organic bases and inorganic or organic acids. Examples of such salts include, but are not limited to, acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide and valerate. Examples of salts derived from inorganic bases include, but are not limited to, aluminum, ammonium, calcium, copper, ferric, ferrous, lithium, magnesium, manganic, mangamous, potassium, sodium, and zinc.

As used herein a wavy line “ [symbol] ” indicates the point of attachment of the bond to the remainder of a molecule.

Certain compounds of present patent application are capable of existing in stereoisomeric forms (e.g. diastereomers and enantiomers) and geometrical forms (E and Z configuration). With respect to the overall compounds described by the general formula (I), the present patent application extends to these stereoisomeric forms, geometrical forms and mixtures thereof. To the extent prior art teaches synthesis or separation of particular stereoisomers, the different stereoisomeric forms of the present patent application may be separated from one another by the method known in the art, or a given isomer may be obtained by stereo specific or asymmetric synthesis. Tautomeric forms and mixtures of compounds described herein are also contemplated. It is also to be understood that compounds of the invention may exist in solvated forms (such as hydrates) as well as un-solvated forms, and that the invention encompasses all such forms.

Accordingly the invention provides a compound of formula (I),

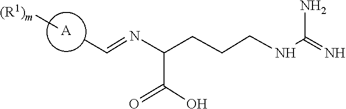

or agrochemically acceptable salt, stereoisomer, enantiomer, tautomer, or N-oxide thereof, wherein,

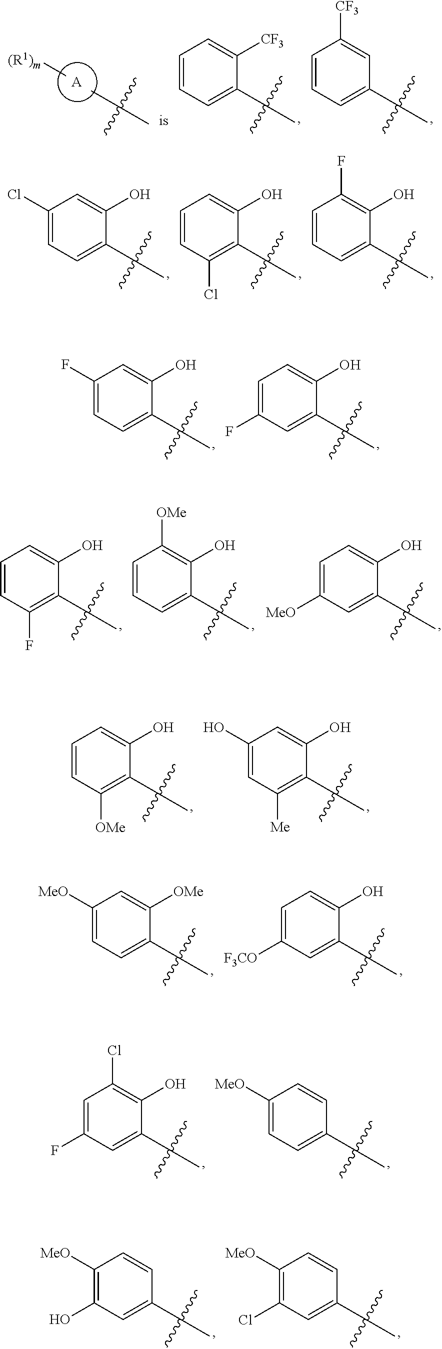

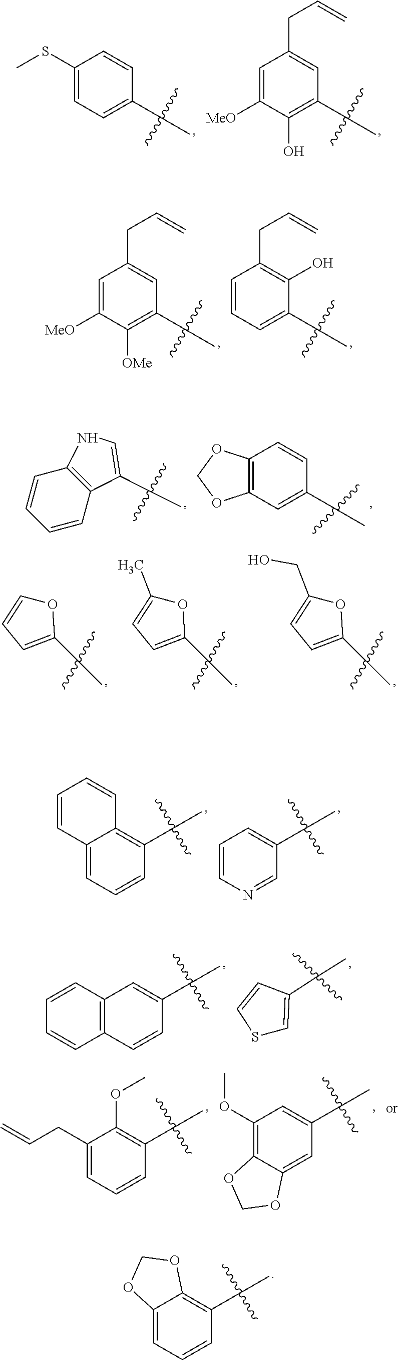

The compounds of the present invention or agrochemically acceptable salt thereof or N-oxide thereof are suitably used on their own but will generally be administered in the form of a composition in which one or more compounds are suitably formulated into compositions, usually using at least one agriculturally acceptable excipient.

Accordingly, the present invention also includes a composition for treating or controlling the parasitic nematode or the nematode infection in a plant comprising an effective amount of one or more compounds of the present invention. In some embodiments, one or more compounds of the present invention are present in an amount that is effective to treat or prevent a nematode infection in a plant.

In an embodiment, the present invention provides compound of formula:

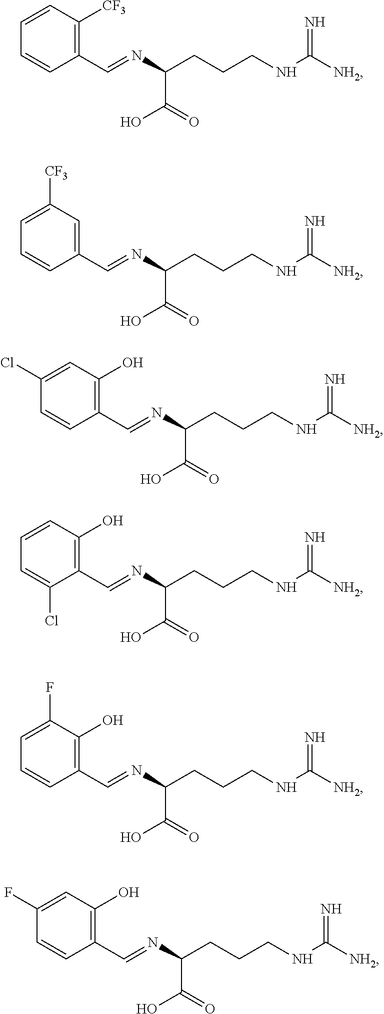

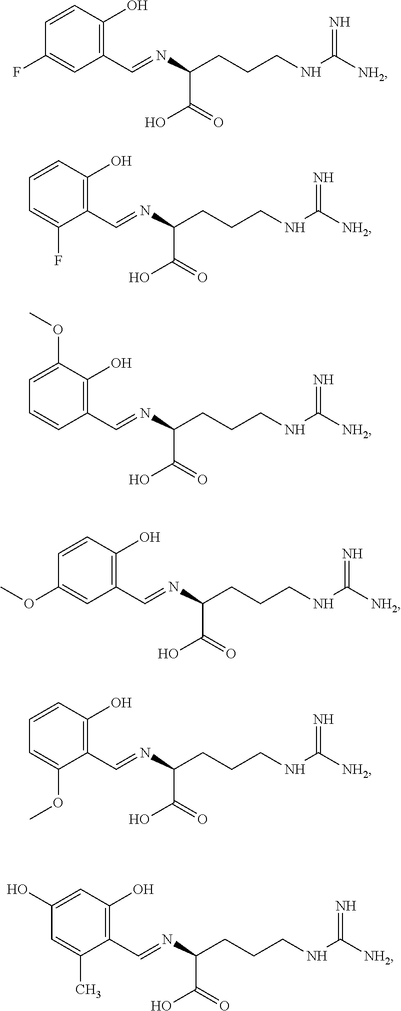 [SMILES: N=C(N)NCCC[C@H](/N=C/c1cc(F)ccc1O)C(=O)O ; N=C(N)NCCC[C@H](/N=C/c1c(O)cccc1F)C(=O)O ; COc1cccc(/C=N/[C@@H](CCCNC(=N)N)C(=O)O)c1O ; COc1ccc(O)c(/C=N/[C@@H](CCCNC(=N)N)C(=O)O)c1 ; COc1cccc(O)c1/C=N/[C@@H](CCCNC(=N)N)C(=O)O ; Cc1cc(O)cc(O)c1/C=N/[C@@H](CCCNC(=N)N)C(=O)O]

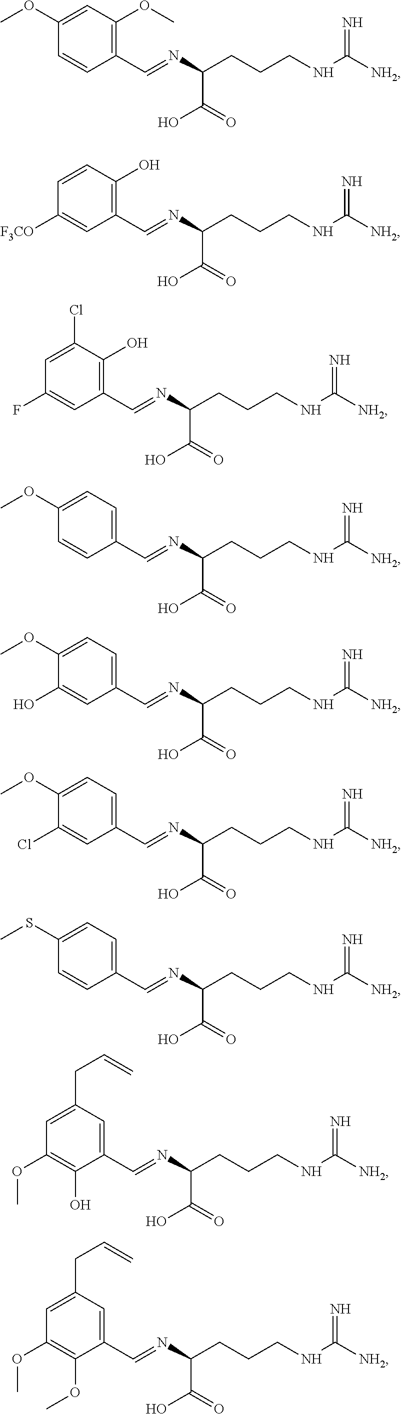

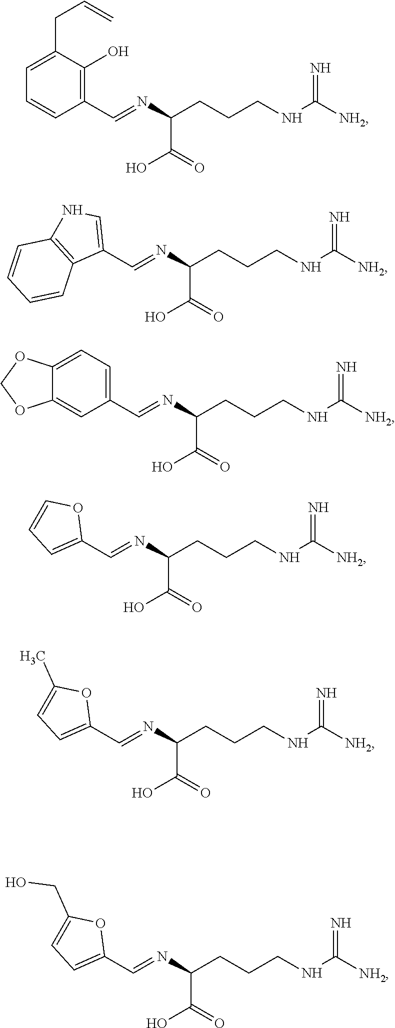 [SMILES: C=CCc1cccc(/C=N/[C@@H](CCCNC(=N)N)C(=O)O)c1O ; N=C(N)NCCC[C@H](/N=C/c1c[nH]c2ccccc12)C(=O)O ; N=C(N)NCCC[C@H](/N=C/c1ccc2c(c1)OCO2)C(=O)O ; N=C(N)NCCC[C@H](/N=C/c1ccco1)C(=O)O ; Cc1ccc(/C=N/[C@@H](CCCNC(=N)N)C(=O)O)o1 ; N=C(N)NCCC[C@H](/N=C/c1ccc(CO)o1)C(=O)O]

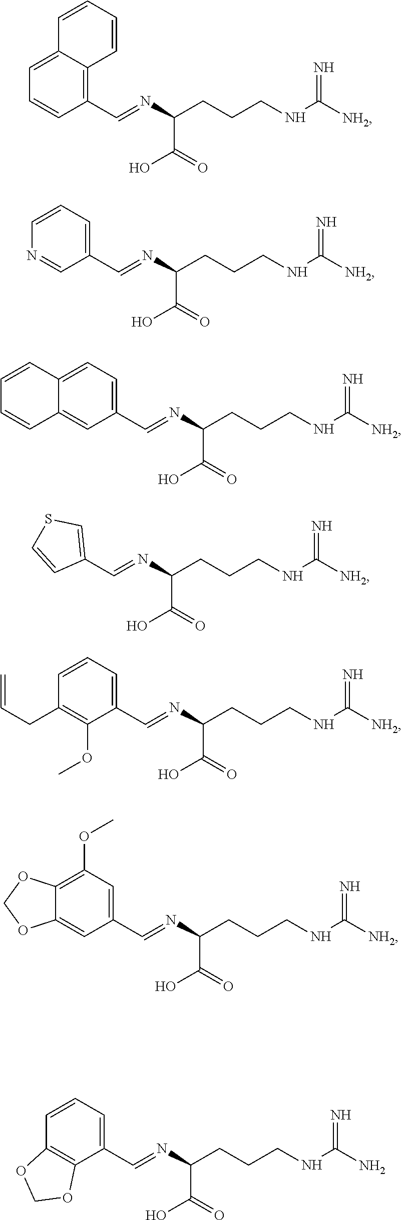

or agrochemically acceptable salt or N-oxide thereof.

In an embodiment, the present invention provides compound of formula:

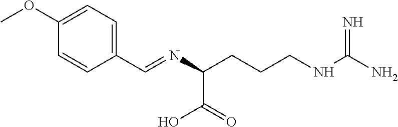 [SMILES: COc1ccc(/C=N/[C@@H](CCCNC(=N)N)C(=O)O)cc1]

or agrochemically acceptable salt or N-oxide thereof.

In an embodiment, the present invention provides compound of formula:

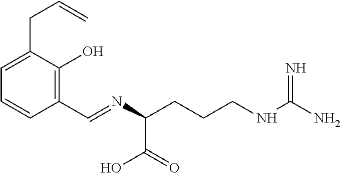 [SMILES: C=CCc1cccc(/C=N/[C@@H](CCCNC(=N)N)C(=O)O)c1O]

or agrochemically acceptable salt or N-oxide thereof.

In an embodiment, the present invention provides compound of formula:

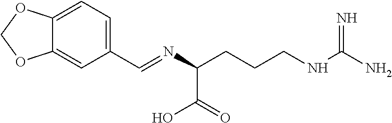 [SMILES: N=C(N)NCCC[C@H](/N=C/c1ccc2c(c1)OCO2)C(=O)O]

or agrochemically acceptable salt or N-oxide thereof.

In an embodiment, the present invention provides compound of formula:

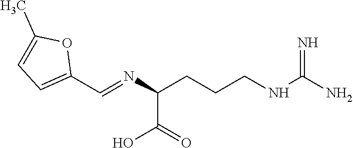 [SMILES: Cc1ccc(/C=N/[C@@H](CCCNC(=N)N)C(=O)O)o1]

or agrochemically acceptable salt or N-oxide thereof.

In an embodiment, the present invention provides an agricultural composition comprising compound of formula (I),

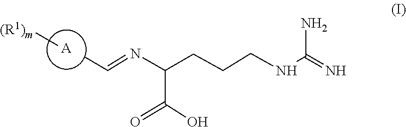

or agrochemically acceptable salt, stereoisomer, enantiomer, tautomer, or N-oxide thereof and at least one agriculturally acceptable excipient, wherein,

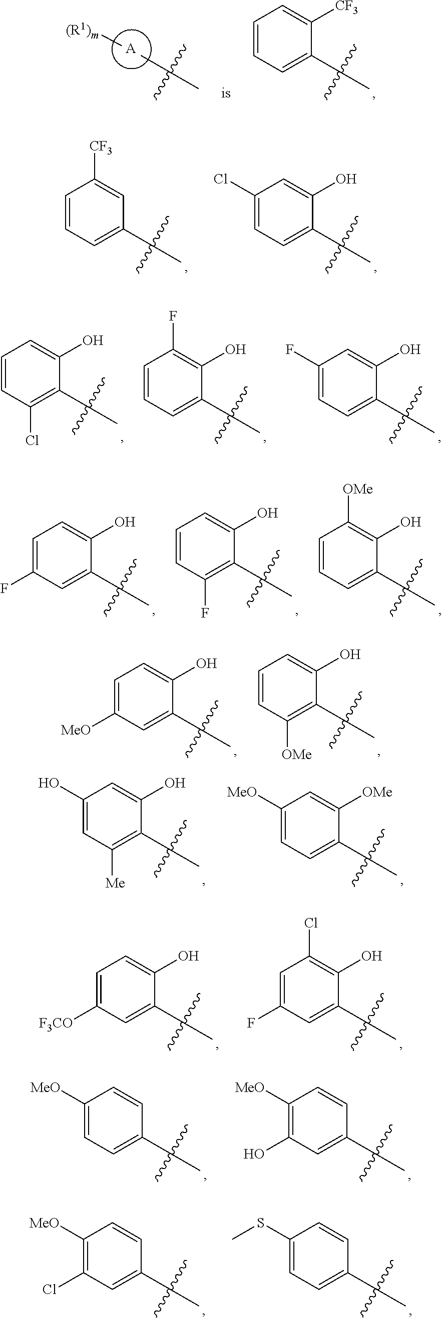

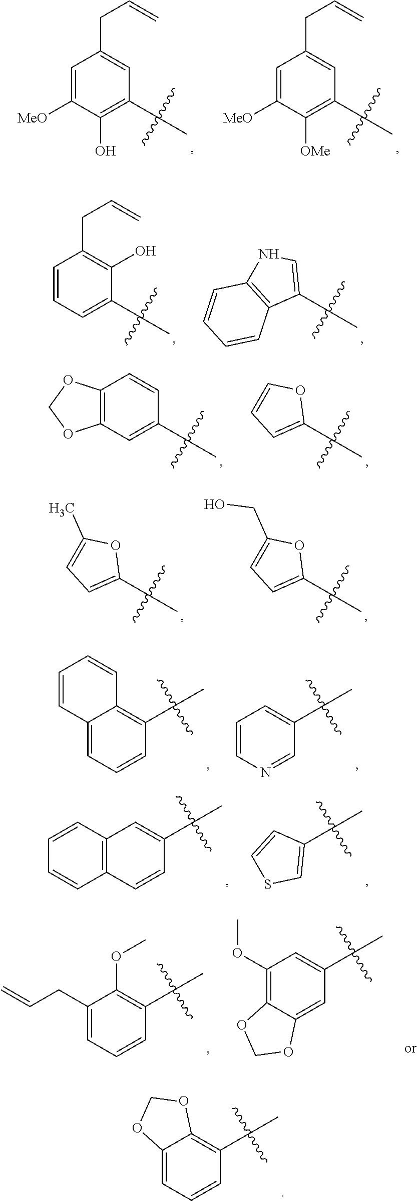

In an embodiment, the present invention provides an agricultural composition comprising compound of formula:

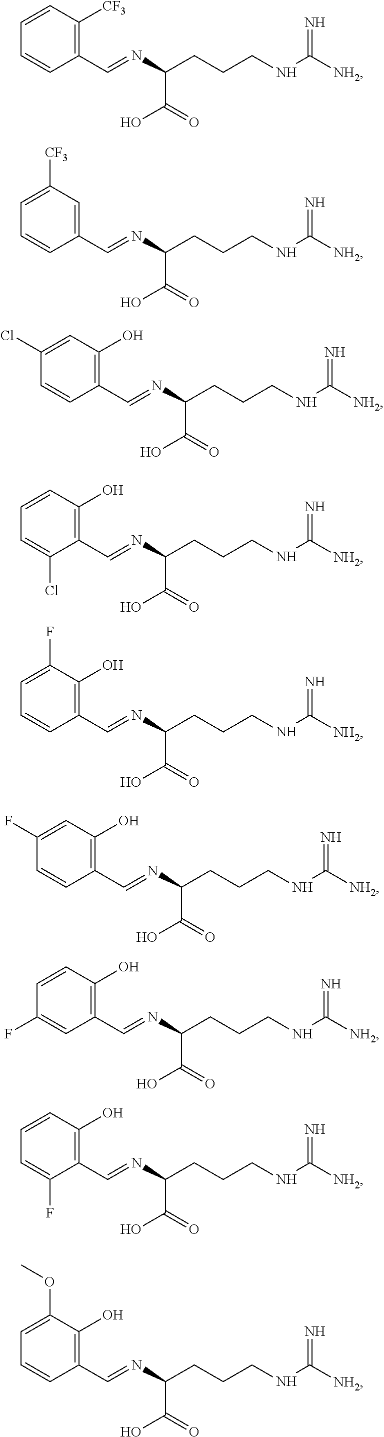

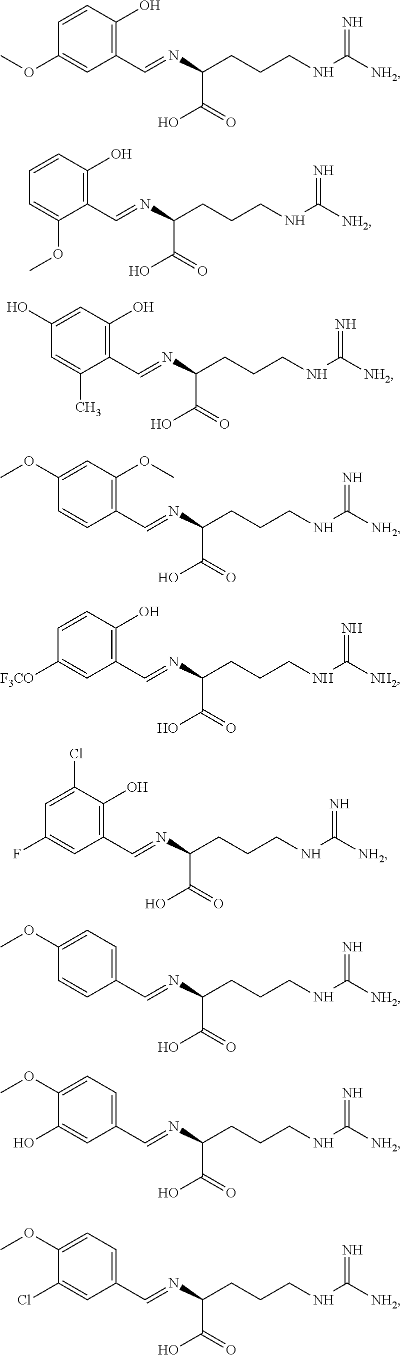

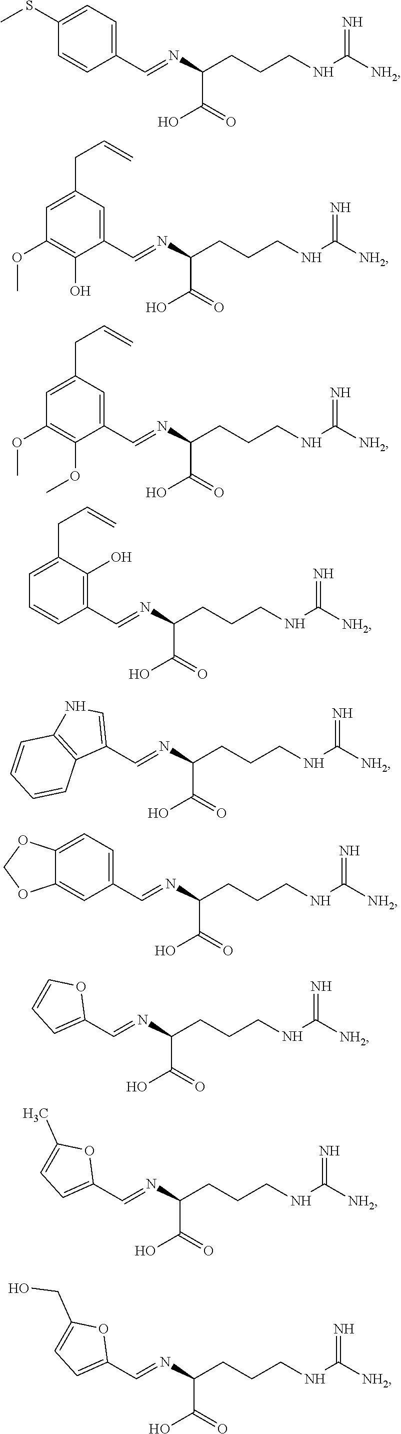

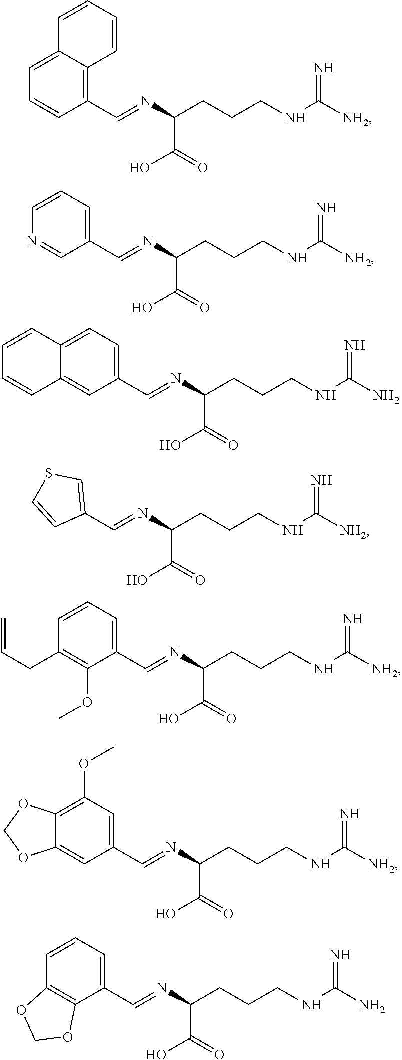

or agrochemically acceptable salt or N-oxide thereof and at least one agriculturally acceptable excipient.

In an embodiment, the present invention provides an agricultural composition comprising a compound of formula:

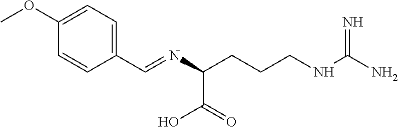 [SMILES: COc1ccc(/C=N/[C@@H](CCCNC(=N)N)C(=O)O)cc1]

or agrochemically acceptable salt or N-oxide thereof, and at least one agriculturally acceptable excipient.

In an embodiment, the present invention provides an agricultural composition comprising a compound of formula:

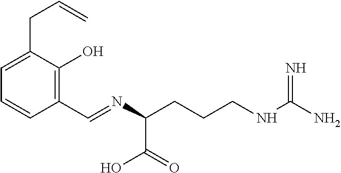 [SMILES: C=CCc1cccc(/C=N/[C@@H](CCCNC(=N)N)C(=O)O)c1O]

or agrochemically acceptable salt or N-oxide thereof, and at least one agriculturally acceptable excipient.

In an embodiment, the present invention provides an agricultural composition comprising a compound of formula:

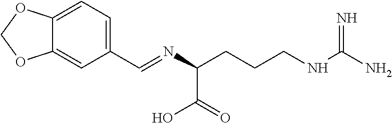 [SMILES: N=C(N)NCCC[C@H](/N=C/c1ccc2c(c1)OCO2)C(=O)O]

or agrochemically acceptable salt or N-oxide thereof, and at least one agriculturally acceptable excipient.

In an embodiment, the present invention provides an agricultural composition comprising a compound of formula:

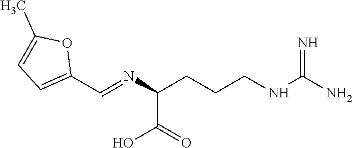 [SMILES: Cc1ccc(/C=N/[C@@H](CCCNC(=N)N)C(=O)O)o1]

or agrochemically acceptable salt or N-oxide thereof, and at least one agriculturally acceptable excipient.

According to an embodiment, the agricultural composition comprises one or more of agrochemically acceptable excipient selected from one or more of surfactants, disintegrating agents, fillers or carriers or diluents, spreading agents, colorants, anticaking agents, binders, buffers or pH adjusters or neutralizing agents, pigments, stabilizers, antifoaming agents or defoamers, penetrants, structuring agents, humectants, sticking agents, anti-freezing agent or freeze point depressants, chelating or complexing or sequestering agents preservatives. However, those skilled in the art will appreciate that it is possible to utilize additional agrochemically acceptable excipients without departing from the scope of the present invention. The agrochemically acceptable excipients are commercially manufactured and available through various companies.

According to an embodiment, the surfactants that are used in the composition of the present invention include one or more of emulsifiers, wetting agents, and dispersing agents. According to an embodiment, the surfactants that are used in the composition include one or more of anionic, non-ionic, and polymeric surfactants. The anionic surfactants include one or more of, but not limited to a salt of Fatty Acid, a Polycarboxylate, Alkyl Ether Sulfates, an Alkyl Sulfate, an Alkylarylsulfate, an Alkylaryl Sulfonate, an Aryl Sulfonate, a Lignin Sulfonate, an Alkyl Diphenyl Ether Disulfonate, a Polystyrene Sulfonate, a Salt of Alkylphosphoric Acid Ester, an Alkylaryl Phosphate, a Styrylaryl Phosphate, a Salt Of Polyoxyethylene Alkyl Ether Sulfuric Acid Ester, Alpha Olefin Sulfonate Sodium Salt, Alkyl Benzene Sulfonate or Its Salts, Sodium Lauroylsarcosinate, Sulfosuccinates, Polyacrylates, Alkyl Ether Phosphate, a Salt of Polyoxyethylenealkylaryl Phosphoric Acid Ester, Sulfosuccinates—Mono and other Diesters, Phosphate Esters, Alkyl Naphthalene Sulfonate-Isopropyl and Butyl Derivatives; Alkyl Aryl Ether Phosphates, a salt of Polyoxyethylene Aryl Ether Phosphoric Acid Ester, Mono-Alkyl Sulphosuccinates, Aromatic Hydrocarbon Sulphonates, Ammonium Laurylsulphate, Soap, Soap Substitute, Sodium Alkyl Sulfate, Sodium Dodecyl Sulfate, Sodium Dodecylbenzenesulfonate, Sodium Laurate, Sodium Laurethsulfate, Sodium Nonanoyloxybenzenesulfonate, Alkyl Carboxylates, Sodium Stearate, Alpha Olefin Sulphonates, Naphthalene Sulfonate Salts, Alkyl Naphthalene Sulfonate Fatty Acid salts, Naphthalene Sulfonate Condensates-Sodium salt, Fatty Alcohol Sulphates, Alkyl Naphthalene Sulfonate Condensates-Sodium Salt, A Naphthalene Sulfonic Acid Condensed with Formaldehyde or a Salt of Alkylnaphthalene Sulfonic Acid condensed with Formaldehyde or salts or derivatives thereof. However, those skilled in the art will appreciate that it is possible to utilize different anionic surfactants without departing from the scope of the present invention.

The non-ionic surfactants or polymeric surfactants include one or more of but not limited to Polyol Esters, Polyol Fatty Acid Esters, Ethoxylated and Propoxylated Fatty Alcohols, EO and PO Block Copolymers, Di, Tri-Block Copolymers, Polysorbates, Alkyl Polysaccharides, Polyoxyethylene Glycol, Sorbitan Derivatives, Fatty Acid Esters of Sorbitan (Spans) and Their Ethoxylated Derivatives (Tweens), Cocamide Monoethanolamine (MEA), Decyl, Narrow-Range Ethoxylate, Oleyl Alcohol, PEG-10, Polysorbate, Polysorbate 20, Polysorbate 80, Sorbitan, Sorbitanmonolaurate, Sorbitanmonostearate, Sorbitantristearate, Stearyl Alcohol, Castor Oil Ethoxylate, Polyglycol Ethers, Polyadducts of Ethylene Oxide and Propylene Oxide, Polyoxy Ethylene Sorbitan, Fatty Acid Polyglyceride, Polyoxyethylene Alkyl Ether, Polyoxyethylenealkylaryl Ether, a Polyoxyethylenestyrylaryl Ether, a Polyoxyethylene Glycol Alkyl Ether, Alcohol Ethoxylates—C6 to C16/18 Alcohols, Linear and Branched, Alcohol Alkoxylates—Various Hydrophobes and EO/PO Contents and Ratios, a Polyoxyethylene Hydrogenated Castor Oil, salts or derivatives thereof. However, those skilled in the art will appreciate that it is possible to utilize different non-ionic surfactants or polymeric surfactants without departing from the scope of the present invention.

According to an embodiment, the dispersing agents which are used in the composition include, but not limited to non-ionic dispersants selected from one or more of polyvinyl pyrrolidone, polyvinyl alcohol, polyoxyethylene alkyl ethers, polyoxyethylene alkyl phenyl ether, ethoxylated fatty acids, aliphatic alcohol ethoxylates, alkyl ethoxylates; EO-PO block and graft copolymers; However, those skilled in the art will appreciate that it is possible to utilize different non-ionic dispersants without departing from the scope of the present invention.

According to an embodiment, the dispersing agents which are used in the composition include, but not limited to anionic dispersants selected from one or, tristyrylphenolethoxylate phosphate esters; lignin sulphonates, phenyl naphthalene sulphonates, alkali metal, alkylarylsulfonates, alkylsulfonates, mixture of sodium salt of naphthalene sulphonic acid urea formaldehyde condensate and sodium salt of phenol sulphonic formaldehyde condensate, polycarboxylates, sodium alkyl benzene sulfonates, sodium salts of sulfonated naphthalene, sodium naphthalene sulfonate formaldehyde condensates, condensation products of aryl sulphonic acids and formaldehyde, polyaromatic sulfonates, sodium alkyl aryl sulfonates and kraft lignin. However, those skilled in the art will appreciate that it is possible to utilize different anionic dispersants without departing from the scope of the present invention.

According to an embodiment the wetting agents used in the composition include, but are not limited to one or more of phenol naphthalene sulphonates, alkyl naphthalene sulfonate, sodium alkyl naphthalene sulfonate, naphthalene sulphonate sodium salt, dibutylnaphthalene-sulfonic acid, alkylarylsulfonates, dioctyl sulfosuccinate, polyoxyethoxylated fatty alcohols, alkane sulfonates, alkylbenzene sulfonates, alkyl ether phosphates, alkyl ether sulphates and alkyl sulfosuccinic monoesters, salts, derivatives thereof. However, those skilled in the art will appreciate that it is possible to utilize different wetting agents without departing from the scope of the present invention.

According to an embodiment, the carriers that are used in the composition of the present invention include, but are not limited to one or more of solid carriers or fillers or diluents. According to another embodiment, the carriers include mineral carriers, plant carriers, synthetic carriers, water-soluble carriers. However, those skilled in the art will appreciate that it is possible to utilize different carriers without departing from the scope of the present invention.

The solid carriers include natural minerals like clay such as china clay, acid clay, kaolin such as kaolinite, dickite, nacrite, and synthetic and diatomaceous silicas, micas, such as pyrophyllite, talc, silicas such as cristobalite and quartz, such as attapulgite and sepiolite, vermiculite, laponite, pumice, bauxite, hydrated aluminas, perlite, sodium bicarbonate, limestone, natural and synthetic silicates, silicas, surface-modified silicas, zeolite, diatomaceous earth, loess, mirabilite, white carbon, slaked lime, synthetic silicic acid, starch, modified starch, cellulose, plant carriers such as cellulose, chaff, wheat flour, wood flour, starch, rice bran, wheat bran, and soybean flour, casein sodium, sucrose, salt cake, potassium pyrophosphate, sodium tripolyphosphate or derivatives or mixtures thereof.

According to an embodiment, the antifoaming agents or defoamers which are used in the composition of the present invention include but are not limited to one or more of silica, siloxane, silicone dioxide, polydimethyl siloxane, alkyl polyacrylates, ethylene oxide/propylene oxide copolymers, silicone oils and magnesium stearate or derivatives thereof. Preferred antifoaming agents include silicone emulsions (such as, e.g., Silikon® SRE, Wacker or Rhodorsil® from Rhodia), long-chain alcohols, fatty acids, fluoro-organic compounds. However, those skilled in the art will appreciate that it is possible to utilize different antifoaming agents without departing from the scope of the present invention.

According to an embodiment, the pH-adjusters or buffers or neutralizing agents that are used in the composition include both acids and bases of the organic or inorganic type and mixtures thereof. According to a further embodiment, pH-adjusters or buffers or neutralizing agents include, but are not limited to one or more of organic acids, inorganic acids, and alkali metal compounds or salts, derivatives thereof. According to an embodiment, the organic acids include, but not limited to one or more of citric, malic, adipic, fumaric, maleic, succinic, and tartaric acids, or salts, derivatives thereof, and the mono-, di-, or tribasic salts of these acids or derivatives thereof. According to an embodiment, the salts of inorganic acids include, but not limited to one or more of alkali metal salts such as, sodium chloride, sodium nitrate, potassium nitrate, sodium sulfate, potassium sulfate, sodium monohydrogen phosphate, potassium monohydrogen phosphate, sodium dihydrogen phosphate, potassium dihydrogen phosphate and the like. Mixtures can also be used to create a pH-adjusters or buffers or neutralizing agents. However, those skilled in the art will appreciate that it is possible to utilize different pH adjusters without departing from the scope of the present invention.

According to an embodiment, the anticaking agents which are used in the composition include, but are not limited to one or more of polysaccharides, fumed and precipitated silica (white carbon), a petroleum resin, Foammaster® Soap L sodium stearate, Brij® 700 polyoxyethylene (100) stearylether, sodium acetate, sodium metasilicate, sodium alkylsulfosuccinates, or derivatives thereof. However, those skilled in the art will appreciate that it is possible to utilize different anticaking agents without departing from the scope of the present invention.

According to an embodiment, the spreading agents which are used in the composition include but are not limited to one or more of copolymer of maleic acid with a styrene compound, a (meth)acrylic acid copolymer, aliphatic alcohols, vegetable oils such as cottonseed or inorganic oils, petroleum distillates, trisiloxanes and modified trisiloxanes, or derivatives thereof. However, those skilled in the art will appreciate that it is possible to utilize different spreading agents without departing from the scope of the present invention.

According to an embodiment, the sticking agents which are used in the composition include, but not limited to one or more of paraffin, a polyamide resin, polyacrylate, polyoxyethylene, wax, latex, polyvinyl pyrrolidone, gums such as xanthan gum, vegetable oils such as cottonseed, or inorganic oils, petroleum distillates, modified trisiloxanes, polyglycol, a synthetic resin emulsion or salts or derivatives thereof. However, those skilled in the art will appreciate that it is possible to utilize different sticking agents without departing from the scope of the present invention.

According to an embodiment, the structuring agents that are used in the composition include, but are not limited to one or more of thickeners, viscosity modifiers, tackifiers, suspension aids, rheological modifiers or anti-settling agents. A structuring agent prevents sedimentation of the active ingredient particles after prolonged storage.

According to an embodiment, the structuring agents which are used in the composition include, but not limited to one or more of polyacrylics, polysaccharides, cellulose derivatives, co-polymers of cellulose derivatives, polyvinyl alcohol and derivatives; clays such as kaolin, smectite, attapulgites and gums such as guar gum, xanthan gum, gelatin, dextrin, fumed silica, mixture of fumed silica and fumed aluminium oxide, swellable polymers, poly(ethylene glycol), stachyose, celluloses such as hemicellulose, carboxymethylcellulose, ethylcellulose, hydroxyethylcellulose, hydroxy-methyl ethyl cellulose, hydroxyl ethyl propyl cellulose, methylhydroxyethylcellulose, methylcellulose; plant starches such as corn starch and potato starch. However, those skilled in the art will appreciate that it is possible to utilize different structuring agents without departing from the scope of the present invention.

Preferred structuring agents include one or more of xanthan gum, aluminum silicate, Hydroxypropyl methylcellulose, carboxymethyl cellulose, methylcellulose, polysaccharide, alkaline earth metal silicate, clays, gelatin, and polyvinyl alcohol.

According to an embodiment, the anti-freezing agents or freezing point depressants used in the composition include, but are not limited to one or more of polyhydric alcohols such as ethylene glycol, diethylene glycol, dipropylene glycol, propylene glycol, glycerol, monohydric or polyhydric alcohols, glycol ethers, glycerol, However, those skilled in the art will appreciate that it is possible to utilize different anti-freezing agents without departing from the scope of the present invention.

According to an embodiment, the chelating or complexing or sequestering agents which are used in the composition include, but not limited to one or more of polycarboxylic acids such as polyacrylic acid and the various hydrolyzed poly(methyl vinyl ether/maleic anhydride); N-hydroxyethyliminodiacetic acid, nitrilotriacetic acid (NTA), N,N,N′,N′-ethylenediaminetetraacetic acid, N-hydroxyethyl-N, N′,N′-ethylenediaminetriacetic acid and N,N,N′,N″,N″-diethylenetriaminepentaacetic acid; α-hydroxy acids, such as citric acid, tartaric acid and gluconic acid; orthophosphates, disodium phosphate, monosodium phosphate; condensed phosphates, such as sodium tripolyphosphate, tetrasodium pyrophosphate, sodium hexametaphosphate and sodium tetrapolyphosphate, ethylene diamine tetraacetic acid (EDTA), diethylenetriaminepentaacetic acid (DTPA), N-hydroxyethyl-ethylenediamine-triacetic acid (HEDTA), ethylenediaminediacetate (EDDA), ethylenediaminedi(o-hydroxyphenylacetic) acid (EDDHA), cyclohexane diamine tetraacetic acid (CDTA), fulvic acid, ulmic acid, nucleic acids, cyclodextrin, humic acid, pyrophosphate. However, those skilled in the art will appreciate that it is possible to utilize different chelating agents without departing from the scope of the present invention.

According to an embodiment, the penetrant which is used in the composition include, but not limited to one or more of alcohol, glycol, glycol ether, ester, amine, alkanolamine, amine oxide, quaternary ammonium compound, triglyceride, fatty acid ester, fatty acid ether, N-methyl pyrrolidone, dimethyl formamide, dimethyl acetamide, or dimethyl sulfoxide, polyoxyethylenetrimethylolpropanemonooleate, polyoxyethylene sorbitan monooleate polyoxyethylenetrimethylolpropanedioleate, polyoxyethylene trimethylol propane trioleate, polyoxyethylene sorbitol hexaoleate. However, those skilled in the art will appreciate that it is possible to utilize different penetrants without departing from the scope of the present invention.

According to an embodiment, the humectant is selected from, but not limited to one or more of polyoxyethylene/polyoxypropylene copolymers, particularly block copolymers. Other humectants are propylene glycol, monoethylene glycol, hexylene glycol, butylene glycol, ethylene glycol, diethylene glycol, poly (ethylene glycol), poly (propylene glycol), glycerol and the like; polyhydric alcohol compounds such as propylene glycol ether, derivatives thereof. However, those skilled in the art will appreciate that it is possible to utilize different humectants without departing from the scope of the present invention.

According to an embodiment, the stabilizers which are used in the agricultural composition include, but not limited to one or more of peroxide compounds such as hydrogen peroxide and organic peroxides, zeolite, antioxidants such as phenol compounds, phosphoric acid compounds, EDTA, sodium sulphites, citric acid, citrates and the like. However, those skilled in the art will appreciate that it is possible to utilize other conventionally known stabilizers without departing from the scope of the present invention.

According to an embodiment, preservative is selected from one or more of formic acid, and derivatives of 2H isothiazol-3-one (so-called isothiazolone derivatives) such as alkylisothiazolones (for example 2-methyl-2H-isothiazol-3-one, MIT; chloro-2-methyl-2H-isothiazol-3-one, CIT), benzoisothiazolones (for example 1,2-benzoisothiazol-3(2H)-one, BIT, commercially available as Proxel® types from Arch Biocides Ltd.) or 2-methyl-4,5-trimethylene-2H-isothiazol-3-one (MTIT), Proxel® from Arch Biocides Ltd. or Acticide® RS from Thor Chemie and Kathon® MK from Lanxess, Sodium Propinoate, Sodium Benzoate, Propyl Paraben, Propyl Paraben Sodium, Potassium Sorbate, Potassium Benzoate, Phenyl Mercuric Nitrate, Phenyl Ethyl Alcohol, Sodium, Ethylparaben, Methylparaben, Butylparaben, Bezyl Alcohol, Benzothonium Chloride, Cetylpyridinium Chloride, Antioxidants includes but not limited to one or more of imidazole and imidazole derivatives (e.g. urocanic acid), 4,4′-thiobis-6-t-butyl-3-methylphenol, 2,6-di-t-butyl-p-cresol (BHT), penta erythrityl tetrakis[3-(3,5-di-t-butyl-4-hydroxyphenyl)]propionate; amine antioxidants. However, those skilled in the art will appreciate that it is possible to utilize other conventionally known preservatives without departing from the scope of the present invention.

According to an embodiment, the pigments and colorants are selected from but not limited to synthetic chemicals obtained from various manufacturers. The pigments and colorants can be water soluble or water insoluble, in the form of lakes. Dyes can be solvent dyes, acid dyes or basic dyes. Examples of such products include, but not limited, Unisperse Red 3855, Pigmosol Agro Red 3785, pigment 15. However, those skilled in the art will appreciate that it is possible to utilize other conventionally known pigments and colorants without departing from the scope of the present invention.

According to an embodiment, the disintegrating agents which are used in the agricultural composition include, but not limited to one or more of inorganic water soluble salts e.g. sodium chloride; water soluble organic compounds such as agar, hydroxypropyl starch, carboxymethyl starch ether, tragacanth, cross-linked sodium carboxymethyl cellulose, sodium tripolyphosphate, sodium hexametaphosphate, metal stearates, a cellulose powder, dextrin, methacrylate copolymer, Polyplasdone® XL-10 (crosslinked polyvinylpyrrolidone), poly(vinylpyrrolidone). However, those skilled in the art will appreciate that it is possible to utilize other conventionally known disintegrating agents without departing from the scope of the present invention.

According to an embodiment, the binding agents or binders that are used in the agricultural composition include, but not limited to one or more of maltodextrin, carbohydrates such as monosaccharides, disaccharides, oligosaccharides and polysaccharides, complex organic substance, synthetic organic polymers or derivatives and combinations thereof. However, those skilled in the art will appreciate that it is possible to utilize other conventionally known binding agents without departing from the scope of the present invention.

According to an embodiment, the agrochemically acceptable excipient is present in the range of from 0.1% w/w to 98% w/w of the total composition. According to a further embodiment, the agrochemically acceptable excipient is present in the range of from 0.1% w/w to 95% w/w of the total composition.

According to an embodiment the agricultural composition is in the form of a solid or a liquid or a gel or a paste.

According to an embodiment, the solid agricultural composition can include powder, granules, and dust.

According to an embodiment, the agricultural composition can be in the form of powder including wettable powder, and dispersible powder. According to an embodiment, the composition can be in the form of granules including broadcast granule, water disintegrable granule, spheronised granule, pellets, extruded granules and water dispersible granules.

According to an embodiment, the composition preferably is in the form of water dispersible granules, wettable powder, broadcast granule, water disintegrable granule or spheronised granule.

According to an embodiment, the composition preferably is solid composition which is in the form of granules including spheronised granules, extruded granules, water disintegrable granules, wettable powders, water dispersible granules, dustable powder (DP), powders for dry seed treatment (DS), water disintegrable tablet or Water Dispersible powders for slurry seed treatment (WS).

According to an embodiment, the liquid composition can include suspension, emulsion, liquid suspension, flowable concentrate, emulsifiable concentrate, seed dressing, suspo-emulsion, emulsions in water. In a further embodiment, in the liquid composition, the compound of the invention may be present in a suspended, emulsified or dissolved form. Examples of particular suitable formulation types are solutions, soluble concentrates (e.g. SL, LS), dispersible concentrates (DC), suspensions and suspension concentrates (e.g. SC, Oil dispersion (OD), OF, FS), emulsifiable concentrates (e.g. EC), emulsions (e.g. EW, EO, ES, micro emulsion (ME), and suspo emulsion (SE), suspension concentrate for seed treatment (FS), Ultra-Low-Volume (ULV).

According to an embodiment, the liquid composition can include liquid suspension or suspension concentrate (SC); suspoemulsion (SE), oil dispersion (OD), flowable concentrate (FC), or a suspension concentrate for seed treatment (FS), Ultra-Low-Volume (ULV).

According to an embodiment, the particle size of the composition is in the range of 0.1 micron to 50 microns. According to an embodiment, the composition is in the form of granules which are in the size range of 0.025 to 6 mm.

According to an embodiment, the composition in the form of spheronised granules or broadcast granules or water disintegrable granules or extruded granules, wherein the granules are in the size range of 0.025 to 6 mm.

According to an embodiment, the granules disperse into particles in the size range of 0.1 micron to 50 microns.

In an embodiment, the present invention provides an agricultural composition comprising compound of the present invention in the range of 1% w/w to 95% w/w of the total composition and at least one agrochemically acceptable excipient in the range of 1% w/w to 99% w/w of the total composition, wherein particles are present in the size range of 0.1 micron to 50 microns.

In an embodiment, the present invention provides an agricultural composition comprising compound of the present invention in the range of 1% w/w to 95% w/w of the total composition, at least one agrochemically acceptable excipient in the range of 1% w/w to 99% w/w of the total composition, wherein particles are present in the size range of 0.1 micron to 50 micron and the granules are in the size range of 0.025 to 6 mm.

According to an embodiment, the present invention relates to a process of preparing an agricultural composition of the present invention comprising a compound of the present invention present in the range of 1% w/w to 95% w/w of the total composition and at least one agrochemically acceptable excipient present in the range of 1% w/w to 99% w/w of the total composition.

In an embodiment, the present invention includes a method of treating or controlling the parasitic nematode or the nematode infection, in a plant comprising administering one or more compounds of the present invention.

In an embodiment, the present invention includes a method of treating or controlling the parasitic nematode or the nematode infection in a plant comprising administering composition of one or more compounds of the present invention.

In another embodiment, the present invention also includes use of one or more compounds of the present invention for treating or controlling the parasitic nematode or the nematode infection in a plant.

In another embodiment, the present invention also includes use of composition of one or more compounds of the present invention for treating or controlling the parasitic nematode or the nematode infection in a plant.

The present invention also includes use of composition of one or more compounds of the present invention for preparation of a medicament for treating or controlling the parasitic nematode or the nematode infection in a plant.

In an embodiment, the present invention includes a method of treating or controlling the parasitic nematode or the nematode infection in a plant comprising administering compound of formula (I)

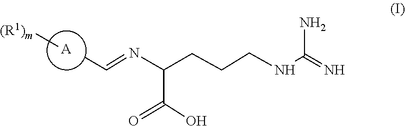

or agrochemically acceptable salt, stereoisomer, enantiomer, tautomer, or N-oxide thereof, wherein,

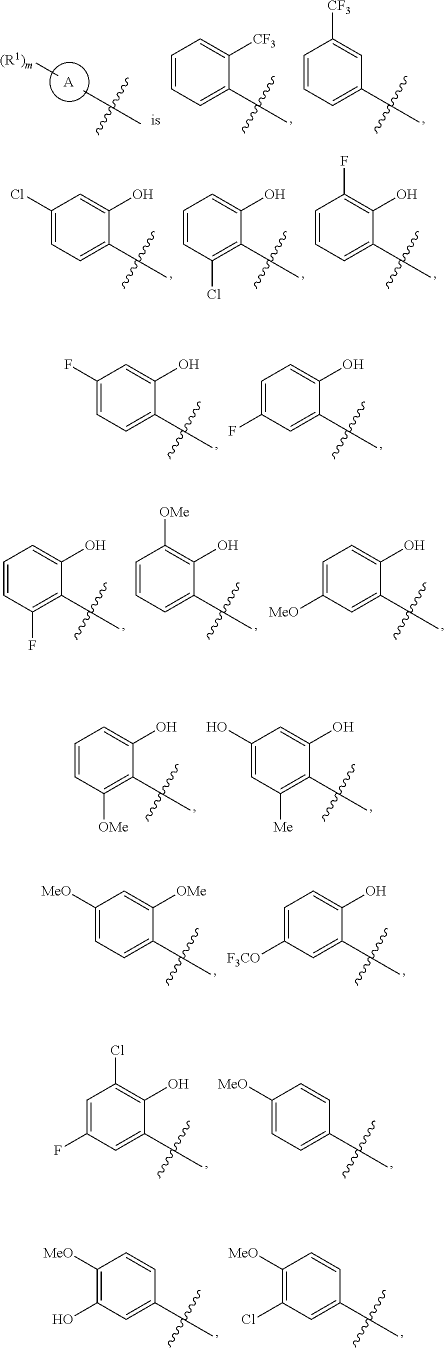

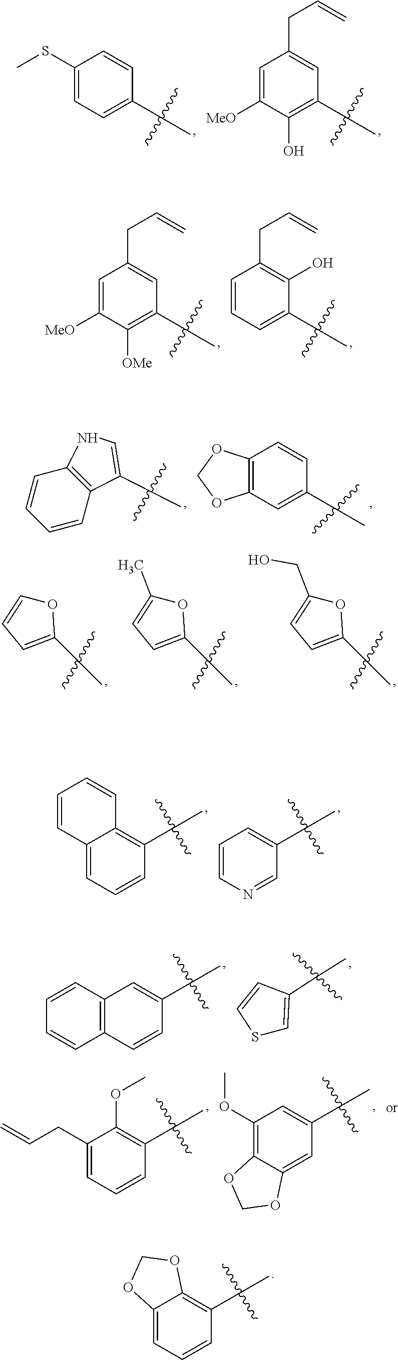

In an embodiment, the present invention includes a method of treating or controlling the parasitic nematode or the nematode infection in a plant comprising administering compound of formula:

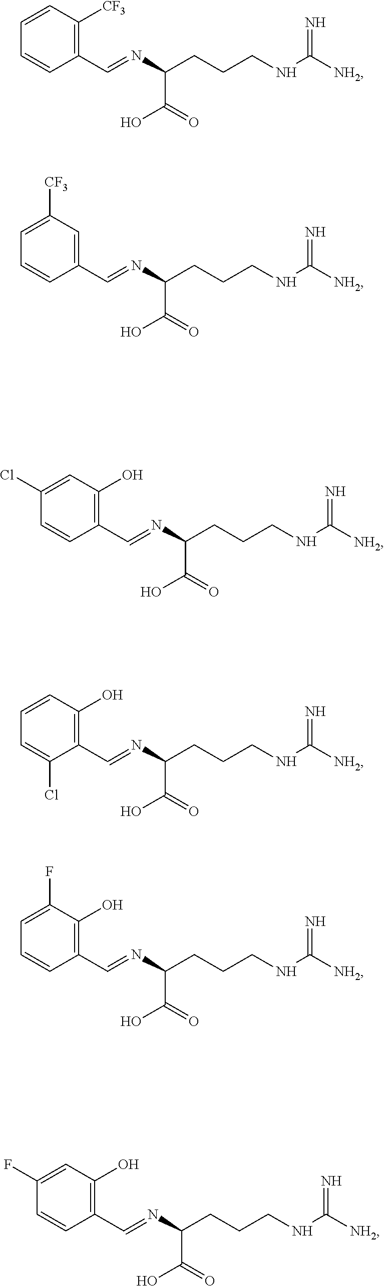 [SMILES: N=C(N)NCCC[C@H](/N=C/c1ccccc1C(F)(F)F)C(=O)O ; N=C(N)NCCC[C@H](/N=C/c1cccc(C(F)(F)F)c1)C(=O)O ; N=C(N)NCCC[C@H](/N=C/c1ccc(Cl)cc1O)C(=O)O ; N=C(N)NCCC[C@H](/N=C/c1c(O)cccc1Cl)C(=O)O ; N=C(N)NCCC[C@H](/N=C/c1cccc(F)c1O)C(=O)O ; N=C(N)NCCC[C@H](/N=C/c1ccc(F)cc1O)C(=O)O]

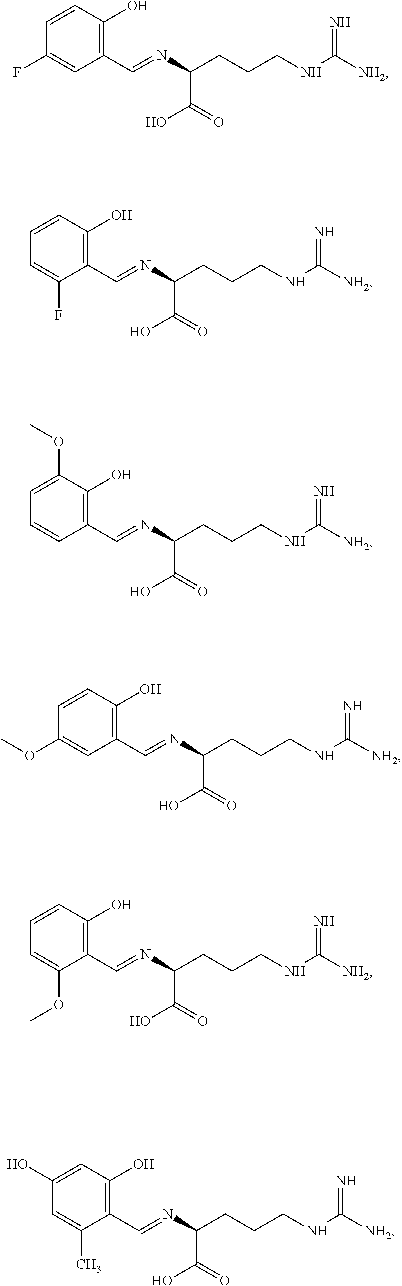 [SMILES: N=C(N)NCCC[C@H](/N=C/c1cc(F)ccc1O)C(=O)O ; N=C(N)NCCC[C@H](/N=C/c1c(O)cccc1F)C(=O)O ; COc1cccc(/C=N/[C@@H](CCCNC(=N)N)C(=O)O)c1O ; COc1ccc(O)c(/C=N/[C@@H](CCCNC(=N)N)C(=O)O)c1 ; COc1cccc(O)c1/C=N/[C@@H](CCCNC(=N)N)C(=O)O ; Cc1cc(O)cc(O)c1/C=N/[C@@H](CCCNC(=N)N)C(=O)O]

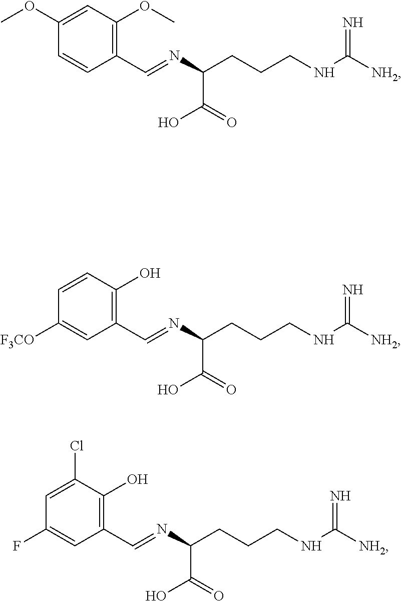 [SMILES: COc1ccc(/C=N/[C@@H](CCCNC(=N)N)C(=O)O)c(OC)c1 ; N=C(N)NCCC[C@H](/N=C/c1cc(OC(F)(F)F)ccc1O)C(=O)O ; N=C(N)NCCC[C@H](/N=C/c1cc(F)cc(Cl)c1O)C(=O)O]

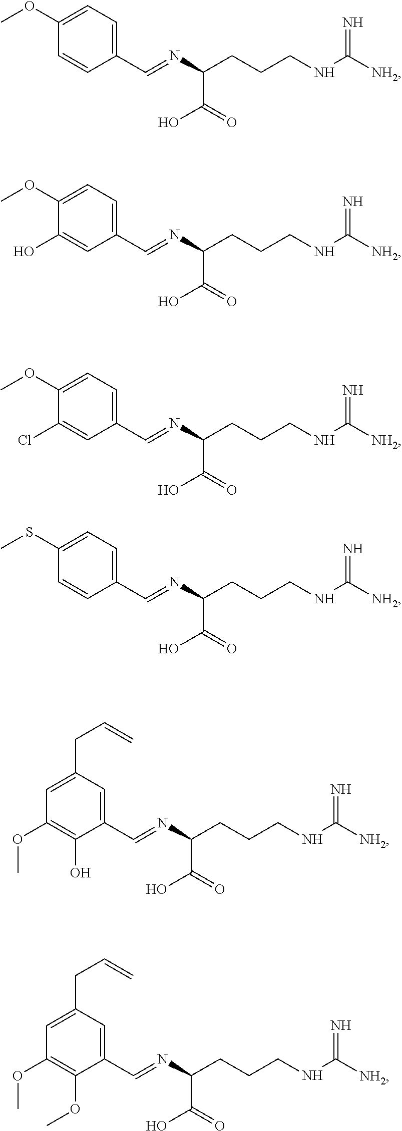 [SMILES: COc1ccc(/C=N/[C@@H](CCCNC(=N)N)C(=O)O)cc1 ; COc1ccc(/C=N/[C@@H](CCCNC(=N)N)C(=O)O)cc1O ; COc1ccc(/C=N/[C@@H](CCCNC(=N)N)C(=O)O)cc1Cl ; CSc1ccc(/C=N/[C@@H](CCCNC(=N)N)C(=O)O)cc1 ; C=CCc1cc(/C=N/[C@@H](CCCNC(=N)N)C(=O)O)c(O)c(OC)c1 ; C=CCc1cc(/C=N/[C@@H](CCCNC(=N)N)C(=O)O)c(OC)c(OC)c1]

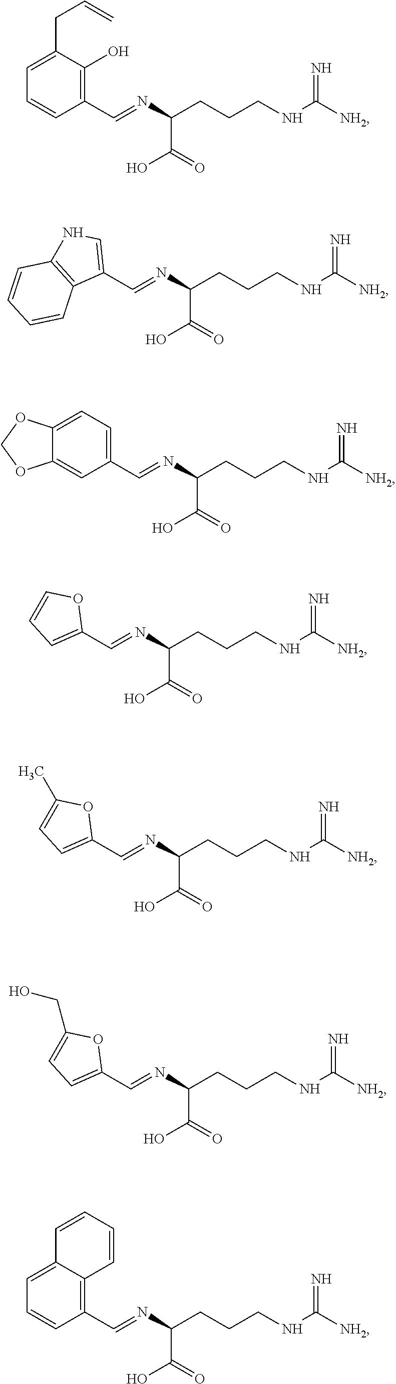

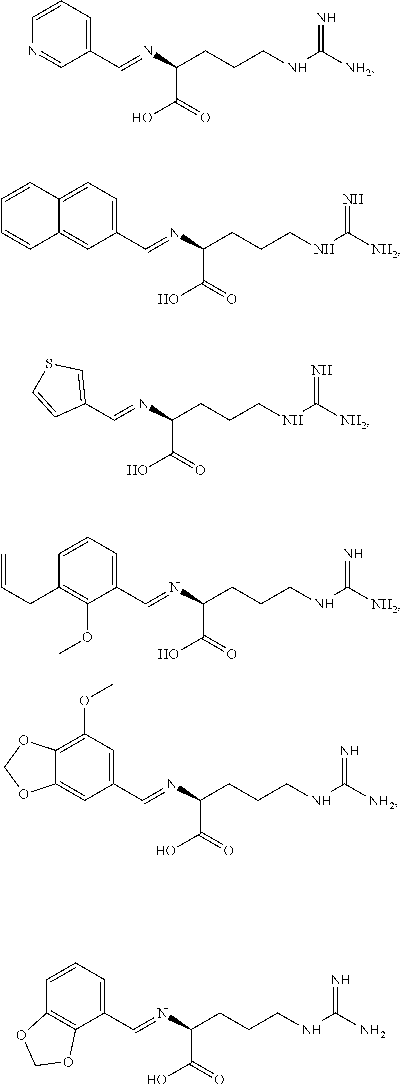 [SMILES: N=C(N)NCCC[C@H](/N=C/c1cccnc1)C(=O)O ; N=C(N)NCCC[C@H](/N=C/c1ccc2ccccc2c1)C(=O)O ; N=C(N)NCCC[C@H](/N=C/c1ccsc1)C(=O)O ; C=CCc1cccc(/C=N/[C@@H](CCCNC(=N)N)C(=O)O)c1OC ; COc1cc(/C=N/[C@@H](CCCNC(=N)N)C(=O)O)cc2c1OCO2 ; N=C(N)NCCC[C@H](/N=C/c1cccc2c1OCO2)C(=O)O]

or agrochemically acceptable salt or N-oxide thereof.

In an embodiment, the present invention includes a method of treating or controlling the parasitic nematode or the nematode infection in a plant comprising administering compound of formula

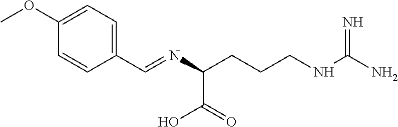 [SMILES: COc1ccc(/C=N/[C@@H](CCCNC(=N)N)C(=O)O)cc1]

or agrochemically acceptable salt or N-oxide thereof.

In an embodiment, the present invention includes a method of treating or controlling the parasitic nematode or the nematode infection in a plant comprising administering compound of formula

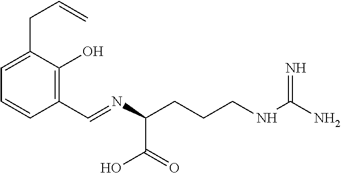 [SMILES: C=CCc1cccc(/C=N/[C@@H](CCCNC(=N)N)C(=O)O)c1O]

or agrochemically acceptable salt or N-oxide thereof.

In an embodiment, the present invention includes a method of treating or controlling the parasitic nematode or the nematode infection in a plant comprising administering compound of formula

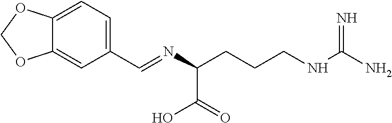 [SMILES: N=C(N)NCCC[C@H](/N=C/c1ccc2c(c1)OCO2)C(=O)O]

or agrochemically acceptable salt or N-oxide thereof.

In an embodiment, the present invention includes a method of treating or controlling the parasitic nematode or the nematode infection in a plant comprising administering compound of formula

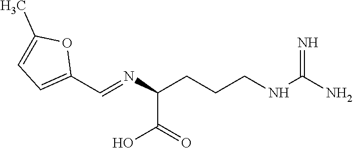 [SMILES: Cc1ccc(/C=N/[C@@H](CCCNC(=N)N)C(=O)O)o1]

or agrochemically acceptable salt or N-oxide thereof.

In another embodiment, the present invention provides use of an agricultural composition for treating or controlling the parasitic nematode or the nematode infection in a plant wherein the composition comprises compound of formula (I),

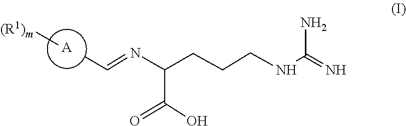

or agrochemically acceptable salt, stereoisomer, enantiomer, tautomer, or N-oxide thereof, and at least one agriculturally acceptable excipient, wherein,

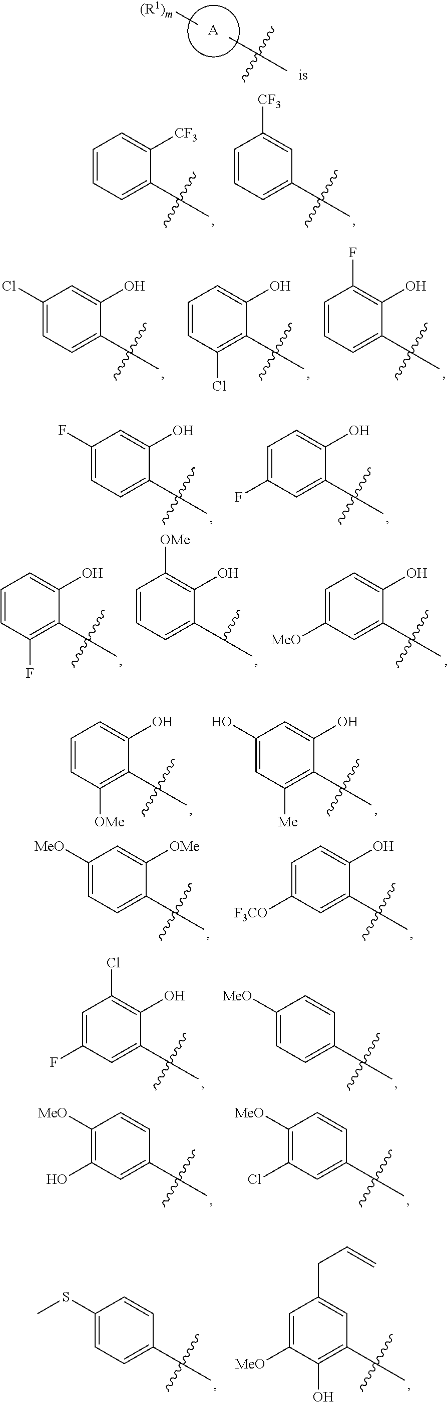

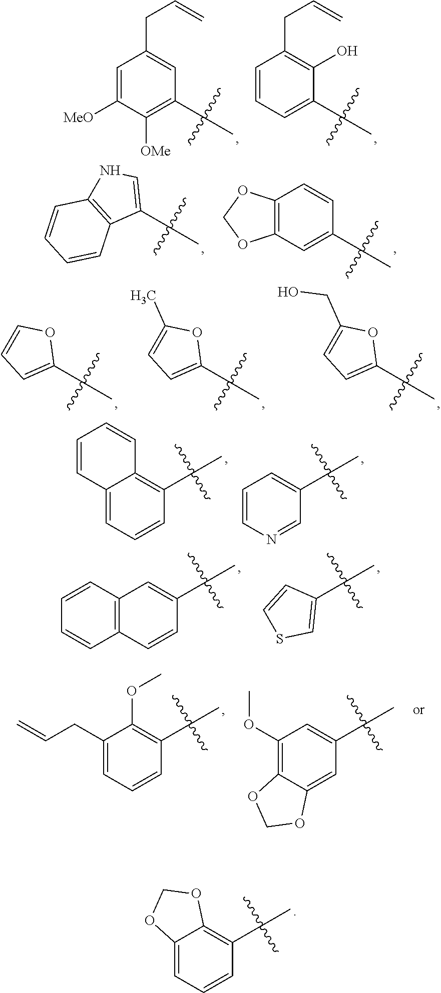

In another embodiment, the present invention provides use of an agricultural composition for treating or controlling the parasitic nematode or the nematode infection in a plant wherein the composition comprises compound of formula:

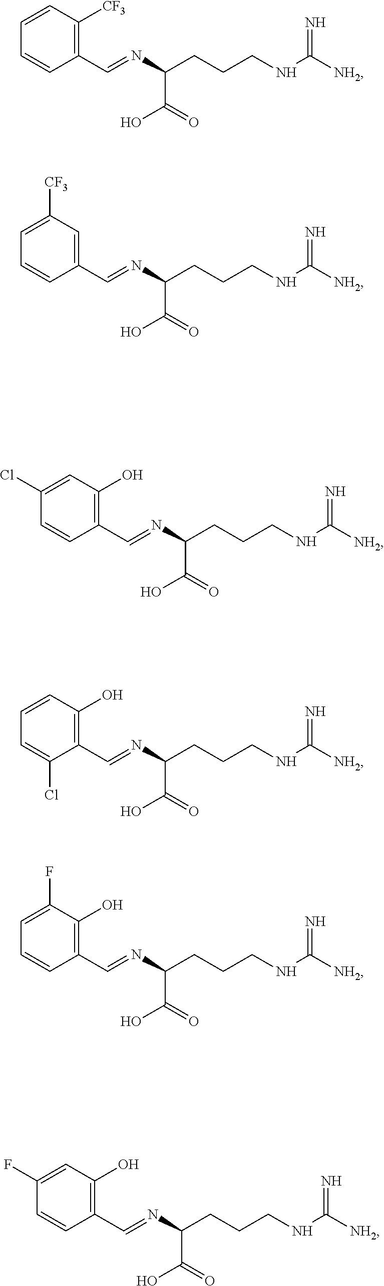 [SMILES: N=C(N)NCCC[C@H](/N=C/c1ccccc1C(F)(F)F)C(=O)O ; N=C(N)NCCC[C@H](/N=C/c1cccc(C(F)(F)F)c1)C(=O)O ; N=C(N)NCCC[C@H](/N=C/c1ccc(Cl)cc1O)C(=O)O ; N=C(N)NCCC[C@H](/N=C/c1c(O)cccc1Cl)C(=O)O ; N=C(N)NCCC[C@H](/N=C/c1cccc(F)c1O)C(=O)O ; N=C(N)NCCC[C@H](/N=C/c1ccc(F)cc1O)C(=O)O]

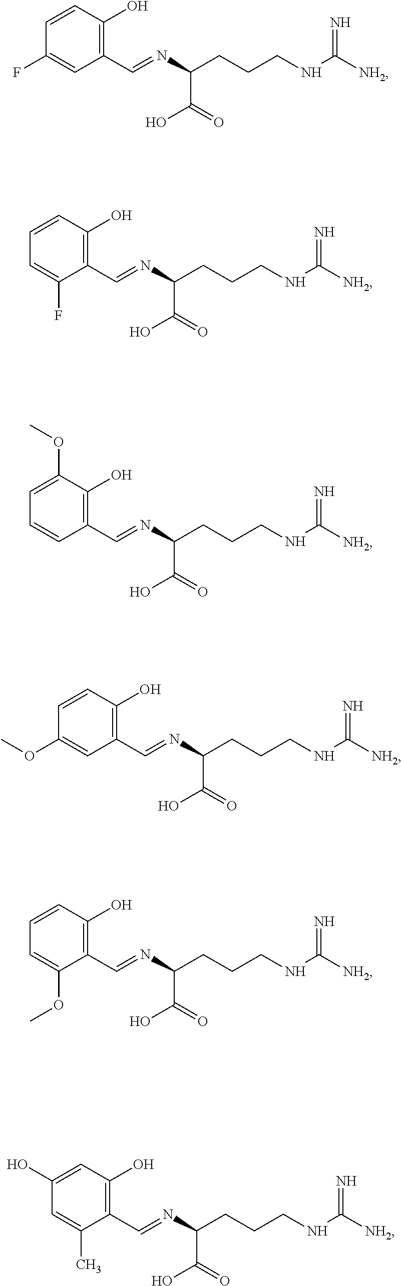 [SMILES: N=C(N)NCCC[C@H](/N=C/c1cc(F)ccc1O)C(=O)O ; N=C(N)NCCC[C@H](/N=C/c1c(O)cccc1F)C(=O)O ; COc1cccc(/C=N/[C@@H](CCCNC(=N)N)C(=O)O)c1O ; COc1ccc(O)c(/C=N/[C@@H](CCCNC(=N)N)C(=O)O)c1 ; COc1cccc(O)c1/C=N/[C@@H](CCCNC(=N)N)C(=O)O ; Cc1cc(O)cc(O)c1/C=N/[C@@H](CCCNC(=N)N)C(=O)O]

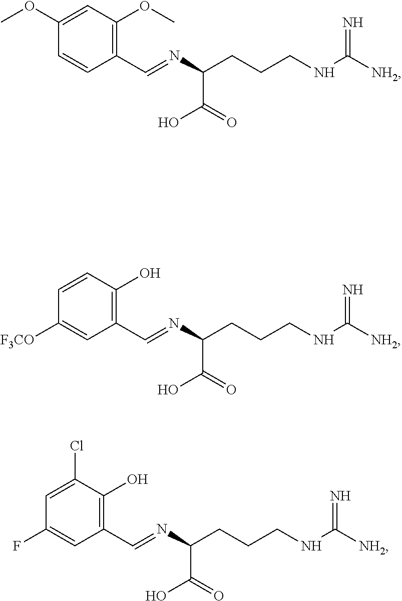 [SMILES: COc1ccc(/C=N/[C@@H](CCCNC(=N)N)C(=O)O)c(OC)c1 ; N=C(N)NCCC[C@H](/N=C/c1cc(OC(F)(F)F)ccc1O)C(=O)O ; N=C(N)NCCC[C@H](/N=C/c1cc(F)cc(Cl)c1O)C(=O)O]

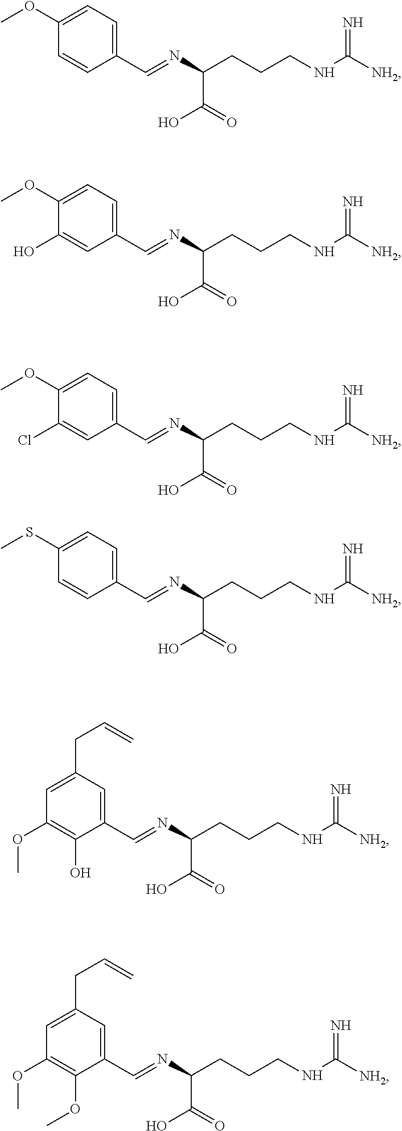 [SMILES: COc1ccc(/C=N/[C@@H](CCCNC(=N)N)C(=O)O)cc1 ; COc1ccc(/C=N/[C@@H](CCCNC(=N)N)C(=O)O)cc1O ; COc1ccc(/C=N/[C@@H](CCCNC(=N)N)C(=O)O)cc1Cl ; CSc1ccc(/C=N/[C@@H](CCCNC(=N)N)C(=O)O)cc1 ; C=CCc1cc(/C=N/[C@@H](CCCNC(=N)N)C(=O)O)c(O)c(OC)c1 ; C=CCc1cc(/C=N/[C@@H](CCCNC(=N)N)C(=O)O)c(OC)c(OC)c1]

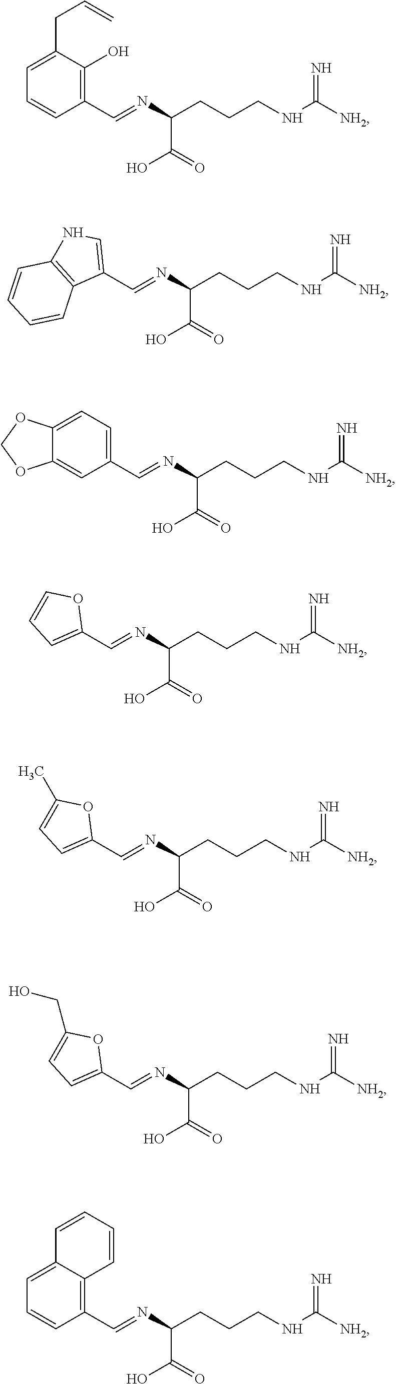

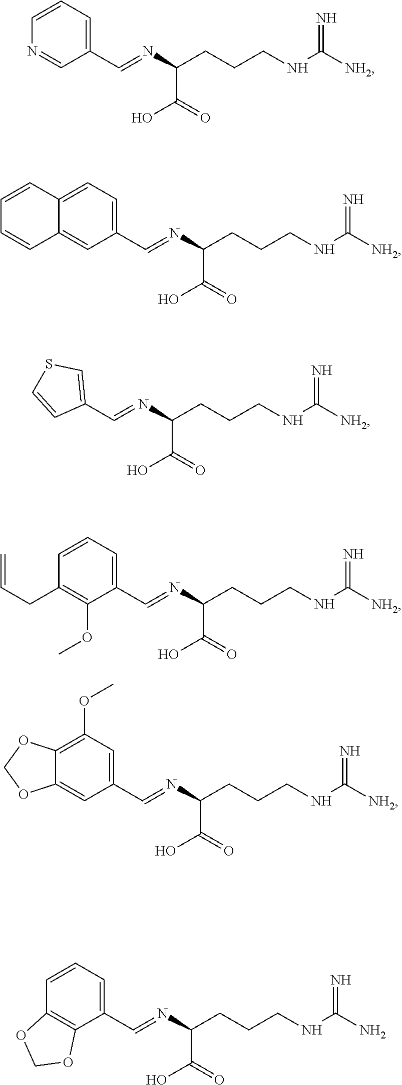 [SMILES: N=C(N)NCCC[C@H](/N=C/c1cccnc1)C(=O)O ; N=C(N)NCCC[C@H](/N=C/c1ccc2ccccc2c1)C(=O)O ; N=C(N)NCCC[C@H](/N=C/c1ccsc1)C(=O)O ; C=CCc1cccc(/C=N/[C@@H](CCCNC(=N)N)C(=O)O)c1OC ; COc1cc(/C=N/[C@@H](CCCNC(=N)N)C(=O)O)cc2c1OCO2 ; N=C(N)NCCC[C@H](/N=C/c1cccc2c1OCO2)C(=O)O]

or agrochemically acceptable salt or N-oxide thereof and at least one agriculturally acceptable excipient.

Nematodes include parasitic nematodes such as root-knot, cyst, and lesion nematodes. The term “nematode” encompasses eggs, larvae, juvenile and mature forms of nematodes. The parasitic nematodes may include but not limited to root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Eelonolaimus longicaudatus and other Belonolaimus species; Pine nematodes, Bursaphelench us xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

In the context of treating or controlling the parasitic nematode or the nematode infection in a plant, an effective amount of one or more compounds of the present invention, is an amount that, for example, reduces the amount of infection by the nematode in the plant compared to the amount of infection by the nematode in the plant without administration of one or more compounds of the application. Reducing the amount of infection may be assessed, for example, by detecting an amount of viable or living nematodes in the plant, and/or by observing or assessing the extent of a disease, disorder or condition caused by a nematode infection.

The dosage of one or more compounds of the present invention in plants, varies depending on many factors such as the pharmacodynamic properties thereof, the mode of administration, the age, health and weight/mass of the plant, the nature and extent of the symptoms, the frequency of the treatment and the type of concurrent treatment, if any. The one or more compounds of the present invention thereof may be administered initially in a suitable dosage that may be adjusted as required, depending on the response.

In some embodiments, one or more compounds of the present invention are applied to plants at any suitable rate, the selection of which can be made by a person skilled in the art. Factors to consider include, for example, the identity of the plant, the identity of the nematode, the identity of the plant disease, disorder or condition, the severity of the nematode infection, the severity of the plant disease, disorder or condition, the age of the plant, the activity of one or more compounds of the application and the concentration of one or more compounds of the application, or a combination thereof.

In some embodiments, the foliage of the plant and/or the soil surrounding the plant is contacted with one or more compounds of the application.

In some embodiments, the nematode infects plants and one or more compounds are administered to the soil or to plants. In some embodiments, one or more compounds are administered to soil before planting. In some embodiments, one or more compounds are administered to soil after planting. In some embodiments, one or more compounds are administered to soil using a drip system. In some embodiments, one or more compounds are administered to soil using a drench system. In some embodiments, one or more compounds are administered to plant roots or plant foliage (e.g., leaves, stems). In some embodiments one or more compounds are tilled into the soil or administered in furrow. In some embodiments, one or more compounds are administered to seeds. In some embodiments, one or more compounds are applied as a seed coating.

In an embodiment, the present invention also provides a method for protecting a seed from a parasitic nematode comprising contacting the seed with an effective amount of a compound of Formula (I) or agrochemically acceptable salt thereof or N-oxide thereof (e.g., as a composition described herein).

Further, the compositions of the present invention are suitable for drip irrigation or sprinkler irrigation in addition to other methods of applications of the agricultural compositions.

According to an embodiment, specifically provided are compounds of formula (I) exhibit greater than 90% reduction of nematode population compared to the untreated control at concentrations such as 100, 200, 250, 300, 500 or 1000 ppm.

According to an embodiment, specifically provided are compounds of formula (I) exhibit greater than 80% reduction of nematode population compared to the untreated control at concentrations such as 100, 200, 250, 300, 500 or 1000 ppm.

According to an embodiment, specifically provided are compounds of formula (I) exhibit greater than 50% reduction of nematode population compared to the untreated control at concentrations such as 100, 200, 250, 300, 500 or 1000 ppm.

In some embodiments, the nematode infects plants and one or more compositions are administered to the soil or to plants. In some embodiments, one or more compositions are administered to soil before planting. In some embodiments, one or more compositions are administered to soil after planting. In an embodiment, one or more compositions are administered to soil using a drip system. In some embodiments, one or more compositions are administered to soil using a drench system. In some embodiments, one or more compositions are administered to plant roots or plant foliage (e.g., leaves, stems). In some embodiments one or more compositions are tilled into the soil or administered in furrow. In some embodiments, one or more compositions are administered to seeds.

In an embodiment, the methods of the application comprise administering one or more compositions of the application through means selected from but not limited to pre-planting, post-planting, as a feed additive, a drench and an external application.

In an embodiment, the term ‘plant’ refers to all physical parts of a plant, including seeds, seedlings, saplings, roots, tubers, stems, stalks, foliage and fruits.

In an embodiment the plant is a cultivated plant. In some embodiments, the plant is an agricultural crop plant. In a further embodiment, the plant includes, but is not limited to, soybeans, cotton, flax, hemp, jute, corn, tobacco, nuts, almonds, coffee, tea, pepper, grapevines, hops, wheat, barley, rye, oats, rice, maize, sorghum, apples, pears, plums, peaches, banana, plantains, cherries, strawberries, raspberries, blackberries, beans, lentils, peas, soya, oilseed rape, mustard, poppies, olives, sunflowers, coconut, castor, cocoa, ground nuts, spinach, asparagus, lettuce, cabbages, carrots, onions, tomatoes, potatoes, bell peppers, cucumbers, melons, pumpkins, sugar cane, sugar beet, fodder beet, avocado, cinnamon, camphor, oranges, tangerines, lemons, limes, grapefruit, latex plants, ornamental plants, and/or turf grasses.

### Methods of Preparation

The compounds described herein, including compounds of general formula (I), and specific examples, are prepared using techniques known to one skilled in the art through the reaction sequences depicted in Scheme 1. Furthermore, in the scheme, where specific bases, reagents, solvents, etc. are mentioned, it is understood that other suitable bases, reagents, etc. may be used and are included within the scope of the present invention. Modifications to reaction conditions, for example, temperature, duration of the reaction or combinations thereof, are envisioned as part of the present invention. The compounds obtained by using the general reaction sequences may be of insufficient purity. These compounds can be purified by using any of the methods for purification of organic compounds known to a person skilled in the art, for example, crystallization or silica gel or alumina column chromatography using different solvents in suitable ratios. All possible stereoisomers are envisioned within the scope of this invention.

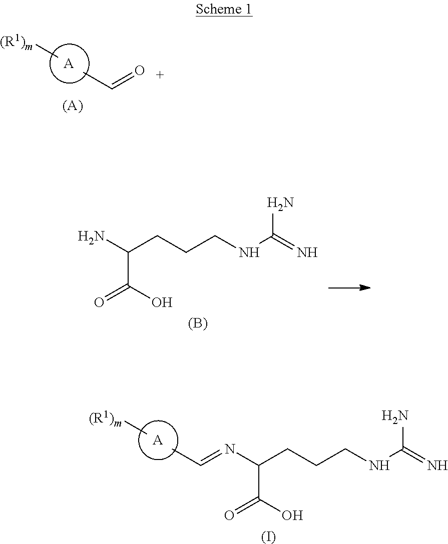

The compounds of the general formula (I) may be prepared as shown in Scheme 1, by reacting the compound of formula (A) or salt thereof with the compound of formula (B) or salt thereof. The reaction can be carried out in the presence of suitable base or it may be carried out in the absence of a base. The reaction may be carried out in the presence of a solvent or mixture of solvents at a suitable temperature range, for example between −10° C. to 100° C. to afford a compound of the general formula (I). The suitable solvents include, but not limited to, water, alcohol (methanol, ethanol, n-propanol, iso-propanol, n-butanol or iso-butanol) tetrahydrofuran, ethyl acetate and acetonitrile or mixture thereof. The suitable bases may include but not limited to organic bases (for example trimethylamine or diisopropylethylamine). The suitable bases may include, but not limited to inorganic bases (for example LiOH, NaOH, NaHCO₃, Na₂CO₃, KOH, KHCO₃, K₂CO₃, Cs₂CO₃ or CsOH).

### EXAMPLES

### Example-1: Preparation of (E)-N²-{[2-(trifluoromethyl)phenyl]methylidene}-L-arginine

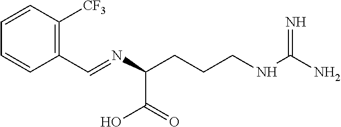 [SMILES: N=C(N)NCCC[C@H](/N=C/c1ccccc1C(F)(F)F)C(=O)O]

2-Trifluromethyl benzaldehyde (1 g) was added to slurry of L-arginine (0.95 g) in ethanol (25 ml) and the mixture was stirred at room temperature for 6 h. The obtained solid was filtered, washed with n-hexane (20 ml) and dried under high vacuum to obtain 1.08 g of ((E)-N2-{[2-(trifluoromethyl)phenyl]methylidene}-L-arginine as a solid. 1H NMR (400 MHz, DMSO-d6) δ 1.40-1.51 (m, 2H), 1.69-1.74 (m, 1H), 1.91-1.94 (m, 1H), 3.08-3.09 (m, 2H), 3.77 (t, J=6.8 Hz, 1H), 7.40-8.00 (brs, 4H), 7.63 (t, J=7.2 Hz, 1H), 7.72 (t, J=7.6 Hz, 1H), 7.78 (d, J=7.6 Hz, 1H), 8.16 (d, J=7.6 Hz, 1H), 8.52 (d, J=2.4 Hz, 1H), 9.44 (s, 1H); ESI-MS: m/z 331.10 (M+H)+.

### Example-2: Preparation of (E)-N²-{[3-(trifluoromethyl)phenyl]methylidene}-L-arginine

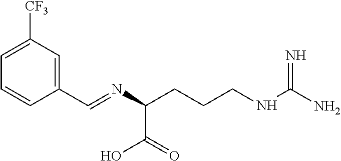 [SMILES: N=C(N)NCCC[C@H](/N=C/c1cccc(C(F)(F)F)c1)C(=O)O]

3-Trifluromethyl benzaldehyde (1 g) was added to slurry of L-arginine (0.95 g) in ethanol (15 ml) and the mixture was stirred at room temperature for 5 h. n-Hexane (15 ml) was added to the mixture and it was stirred for further 2 hrs at room temperature. The obtained solid was filtered, washed with n-hexane (10 ml) and dried under high vacuum to get 1.59 g of ((E)-N²-{[3-(trifluoromethyl)phenyl]methylidene}-L-arginine as a solid. ¹H NMR (400 MHz, DMSO-d₆) δ 1.47-1.52 (m 2H), 1.68-1.73 (m, 1H), 1.90-1.94 (m, 1H), 3.80-3.12 (m, 2H), 3.75 (t, J=6.8 Hz, 1H), 7.20-7.90 (br s, 4H), 7.71 (t, J=7.6 Hz, 1H), 7.77 (d, J=8.0 Hz, 1H), 8.01 (d, J=7.6 Hz, 1H), 8.05 (s, 1H), 8.35 (s, 1H), 9.39 (br s, 1H); ESI-MS: m/z 331.10 (M+H)+.

### Example-3: Preparation of (E)-N²-[(4-chloro-2-hydroxyphenyl)methylidene]-L-arginine

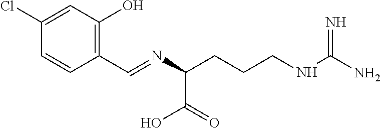 [SMILES: N=C(N)NCCC[C@H](/N=C/c1ccc(Cl)cc1O)C(=O)O]

L-arginine (1.07 g) was added to the solution of 4-chloro2-hydroxybenzaldehyde (1.0 g) in ethanol (20 ml) and the reaction mixture was stirred at RT for 18 h. The obtained solid was filtered, washed with n-hexane (20 ml) and dried under high vacuum to get 1.28 g of (E)-N²-[(4-chloro-2-hydroxyphenyl)methylidene]-L-arginine as a solid. ¹H NMR (400 MHz, DMSO-d₆) δ 1.44-1.51 (m, 2H), 1.75-1.91 (m, 2H), 3.08-3.10 (m, 2H), 3.91 (t, J=6.4 Hz, 1H), 6.54 (dd, J=2.0, 6.4 Hz, 1H), 6.65 (d, J=1.6 Hz, 1H), 7.28 (d, J=8.4 Hz, 1H), 7.44 (brs, 4H), 8.41 (brs, 1H), 9.39 (brs, 1H), 14.53 (brs, 1H); ESI-MS: m/z 313.10 (M+H)⁺.

### Example-4: Preparation of (E)-N²-[(6-chloro-2-hydroxyphenyl)methylidene]-L-arginine

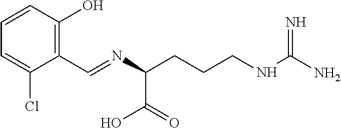 [SMILES: N=C(N)NCCC[C@H](/N=C/c1c(O)cccc1Cl)C(=O)O]

L-arginine (1.07 g) was added to the solution of 6-chloro-2-hydroxybenzaldehyde (1.0 g) in ethanol (20 ml) and reaction mixture was stirred at RT for 4 h. The solid was filtered, washed with n-hexane (20 ml) and dried under high vacuum to give 1.64 g of (E)-N²-[(6-chloro-2-hydroxyphenyl)methylidene]-L-arginine as solid. ¹H NMR (400 MHz, DMSO-d₆) δ 1.45-1.54 ((m, 2H), 1.76-1.95 (m, 2H), 3.10-3.16 (m, 2H), 4.06 (t, J=6.4 Hz, 1H), 6.64-6.67 (m, 2H), 7.22 (dd, J=0.4, 8.0 Hz, 1H), 7.55 (brs, 4H), 8.72 (d, J=5.6 Hz, 1H), 9.33 (brs, 1H), 15.25 (brs, 1H); ESI-MS: m/z 313.50 (M+H)⁺.

### Example-5: Preparation of (E)-N²-[(3-fluoro-2-hydroxyphenyl)methylidene]-L-arginine

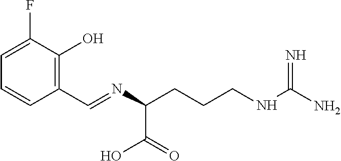 [SMILES: N=C(N)NCCC[C@H](/N=C/c1cccc(F)c1O)C(=O)O]

3-fluoro-2-hydroxy benzaldehyde (1 g) was added to slurry of L-arginine (1.18 g) in ethanol (20 ml) and the mixture was stirred at RT for 4 h. The obtained solid was filtered, washed with n-hexane (20 ml) and dried under high vacuum to give 1.54 g (E)-N²-[(3-fluoro-2-hydroxyphenyl)methylidene]-L-arginine as a solid. ¹H NMR (400 MHz, DMSO-d₆) δ 1.46-1.76 (m, 2H), 1.78-1.96 (m, 2H), 3.10-3.20 (m, 2H), 3.96 (t, J=6.8 Hz, 1H), 6.34-6.39 (m, 1H), 7.04-7.48 (m, 2H), 7.25-8.0 (brs, 4H), 8.44 (s, 1H), 9.35 (s, 1H), 14.40 (brs, 1H); ESI-MS: m/z 297.20 (M+H)+.

### Example-6: Preparation of (E)-N²-[(4-fluoro-2-hydroxyphenyl)methylidene]-L-arginine

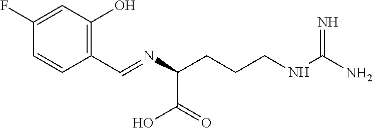 [SMILES: N=C(N)NCCC[C@H](/N=C/c1ccc(F)cc1O)C(=O)O]

4-fluoro-2-hydroxy-benzaldehyde (1 g) was added to the slurry of L-arginine (1.18 g) in ethanol (25 ml) and stirred at RT for 3 h under nitrogen atmosphere. Then reaction mixture was filtered, washed with n-hexane (10 ml) and dried under high vacuum to give 1.36 g (E)-N²-[(4-fluoro-2-hydroxyphenyl)methylidene]-L-arginine as a solid. ¹H NMR (400 MHz, DMSO-d₆) δ 1.48-1.51 (m, 2H), 1.76-1.90 (m, 2H), 3.09-3.11 (m, 2H), 3.91 (t, J=6.0 Hz, 1H), 6.28-6.35 (m, 2H), 7.29 (t, J=8.4 Hz, 1H), 7.20-7.80 (br s, 4H), 8.36 (s, 1H), 9.36 (s, 1H), 14.45 (s, 1H); ESI-MS: m/z 297.10 (M+H)+.

### Example-7: Preparation of (E)-N²-[(5-fluoro-2-hydroxyphenyl)methylidene]-L-arginine

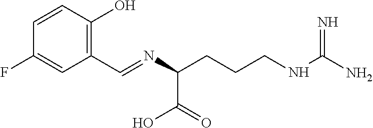 [SMILES: N=C(N)NCCC[C@H](/N=C/c1cc(F)ccc1O)C(=O)O]

5-fluoro-2-hydroxy benzaldehyde (1 g) was added to slurry of L-arginine (1.18 g) in ethanol (20 ml) and the mixture was stirred at RT for 5 h. Then solid was filtered, washed with n-hexane (20 ml) and dried under high vacuum to give 1.57 g (E)-N²-[(5-fluoro-2-hydroxyphenyl)methylidene]-L-arginine as a solid. ¹H NMR (400 MHz, DMSO-d₆) δ 1.46-1.51 (m, 2H), 1.70-1.76 (m, 1H), 1.89-1.93 (m, 1H), 3.07-3.09 (m, 2H), 3.78 (t, J=6.4 Hz, 1H), 6.80-6.84 (m, 1H), 7.12-7.18 (m, 1H), 7.26-7.29 (m, 1H), 7.30-7.90 (brs, 4H), 8.42 (s, 1H), 9.57 (s, 1H), 14.06 (s, 1H); ESI-MS: m/z 297.40 (M+H)+.

### Example-8: Preparation of (E)-N²-[(6-fluoro-2-hydroxyphenyl)methylidene]-L-arginine

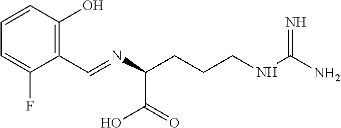 [SMILES: N=C(N)NCCC[C@H](/N=C/c1c(O)cccc1F)C(=O)O]

6-fluoro-2-hydroxy-benzaldehyde (1 g) was added to the slurry of L-arginine (1.18 g) in ethanol (25 ml) and it was stirred at RT for 3 h under nitrogen atmosphere. The solid was filtered, washed with n-hexane (10 ml) and dried under high vacuum to give 1.61 g (E)-N²-[(6-fluoro-2-hydroxyphenyl)methylidene]-L-arginine as a solid. ¹H NMR (400 MHz, DMSO-d₆) 1.46-1.49 (m, 2H), 1.74-1.80 (m, 1H), 1.87-1.94 (m, 1H), 3.09-3.11 (m, 2H), 4.00 (t, J=6.0 Hz, 1H), 6.31-6.36 (dd, J=7.6, 2.4 Hz, 1H), 6.48 (d, J=8.8 Hz, 1H), 7.23 (dd, J=8.4, 7.6 Hz, 1H), 7.42 (br s, 4H), 8.60 (d, J=4.8 Hz, 1H), 9.40 (s, 1H), 14.75 (s, 1H); ESI-MS: m/z 297.40 (M+H)₊.

### Example-9: Preparation of (E)-N²-[(2-hydroxy-3-methoxyphenyl) methylidene]L-arginine

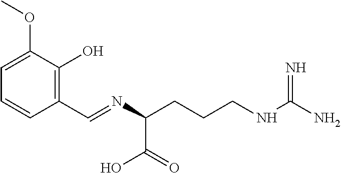 [SMILES: COc1cccc(/C=N/[C@@H](CCCNC(=N)N)C(=O)O)c1O]

ortho-Vanillin (4.59 g) was added to slurry of L-arginine (5.0 g) in ethanol (25 ml) and it was stirred at RT for 6 h under nitrogen atmosphere. N-hexane (50 ml) was added to the reaction mixture and it was stirred for 1 hr at RT. The product was filtered, washed with n-hexane (50 ml) and dried under high vacuum to give 9.10 g of ((E)-N²-[(2-hydroxy-3-methoxyphenyl)methylidene]-L-arginine as a solid. ¹H NMR (400 MHz, DMSO-d₆) δ 1.49-1.59 (m, 2H), 1.71-1.80 (m, 1H), 1.88-1.93 (m, 1H), 3.05-3.15 (m, 2H), 3.73 (s, 3H), 3.85 (t, J=6.4 Hz, 1H), 6.55 (t, J=8.0 Hz, 1H), 6.87-6.89 (m, 2H), 7.20-8.01 (br s, 4H), 8.36 (s, 1H), 9.53 (br s, 1H), 14.25 (br s, 1H); ESI-MS: m/z 309.10 (M+H)⁺.

### Example-10: Preparation of (E)-N²-[(2-hydroxy-5-methoxyphenyl)methylidene]-L-arginine

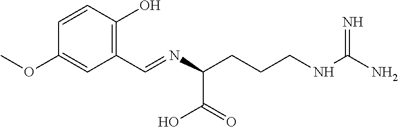 [SMILES: COc1ccc(O)c(/C=N/[C@@H](CCCNC(=N)N)C(=O)O)c1]

L-arginine (1.09 g) was added to the solution of 2-hydroxy-5-methoxybenzaldehyde (1.0 g) in ethanol (25 ml) and the reaction mixture was stirred at RT for 2 h. The solid was filtered, washed with n-hexane (25 ml) and dried under high vacuum to give 1.57 g (E)-N²-[(2-hydroxy-5-methoxyphenyl)methylidene]-L-arginine as a solid. ¹H NMR (400 MHz, DMSO-d₆) δ 1.46-1.51 (m, 2H), 1.69-1.74 (m, 1H), 1.90-1.95 (m, 1H), 3.05-3.10 (m, 2H), 3.33 (s, 3H), 3.75 (t, J=6.4 Hz, 1H), 6.76 (d, J=8.8 Hz, 1H), 6.90 (dd, J=2.8, 6.0 Hz, 1H), 7.00 (s, 1H), 7.44 (brs, 4H), 8.40 (s, 1H), 9.51 (brs, 1H), 13.56 (s, 1H); ESI-MS: m/z 309.20 (M+H)⁺.

### Example-11: Preparation of (E)-N²-[(2-hydroxy-6-methoxyphenyl)methylidene]-L-arginine

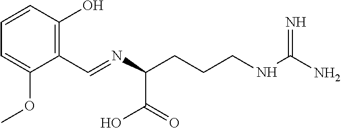 [SMILES: COc1cccc(O)c1/C=N/[C@@H](CCCNC(=N)N)C(=O)O]

L-arginine (1.09 g) was added to the solution of 2-hydroxy-6-methoxybenzaldehyde (1.0 g) in ethanol (25 ml) and the reaction mixture was stirred at RT for 18 h. The solid was filtered, washed with n-hexane (25 ml) and dried under high vacuum to give 1.03 g (E)-N²-[(2-hydroxy-6-methoxyphenyl)methylidene]-L-arginine as a solid. ¹H NMR (400 MHz, DMSO-d₆) δ 1.40-1.51 (m, 2H), 1.68-1.77 (m, 1H), 1.85-1.90 (m, 1H), 3.00-3.10 (m, 2H), 3.78 (s, 3H), 3.88 (t, J=6.4 Hz, 1H), 6.12 (d, J=8.0 Hz, 1H), 6.24 (d, J=8.8 Hz, 1H), 7.16 (t, J=8.0 Hz, 1H), 7.60 (brs, 4H), 8.59 (s, 1H), 9.47 (brs, 1H), 14.84 (brs, 1H); ESI-MS: m/z 309.10 (M+H)⁺.

### Example-12: Preparation of (E)-N²-[(2,4-dihydroxy-6-methylphenyl)methylidene]-L-arginine

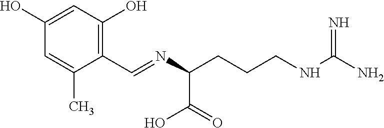 [SMILES: Cc1cc(O)cc(O)c1/C=N/[C@@H](CCCNC(=N)N)C(=O)O]

L-arginine (1.09 g) was added to the solution of 2,4-dihydroxy-6-methylbenzaldehyde (1.0 g) in ethanol (20 ml) and the reaction mixture was stirred at RT for 12 h. The solid was filtered, washed with n-hexane (25 ml) and dried under high vacuum to give 0.91 g (E)-N²-[(2,4-dihydroxy-6-methylphenyl)methylidene]-L-arginine as a solid. ¹H NMR (400 MHz, DMSO-d₆) δ 1.45-1.55 (m, 2H), 1.68-1.95 (m, 2H), 2.20 (s, 3H), 3.05-3.14 (m, 2H), 3.91 (t, J=6.4 Hz, 1H), 6.50 (s, 1H), 7.01 (s, 1H), 7.60 (brs, 4H), 8.59 (s, 1H), 9.47 (brs, 1H), 14.84 (brs, 1H); ESI-MS: m/z 309.20 (M+H)⁺.

### Example 13: Preparation of (E)-N²-[(2,4-dimethoxyphenyl)methylidene]-L-arginine

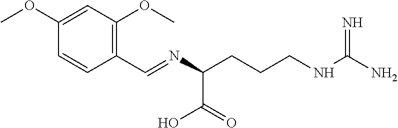 [SMILES: COc1ccc(/C=N/[C@@H](CCCNC(=N)N)C(=O)O)c(OC)c1]

L-arginine (1 g) was added to the solution of 2,4-dimethoxy benzaldehyde (1.0 g) in ethanol (25 ml) and the reaction mixture was stirred at RT for 16 h. The solid was filtered, washed with n-hexane (50 ml) and dried under high vacuum to give 1.70 g of (E)-N²-[(2,4-dimethoxyphenyl)methylidene]-L-arginine as a solid. ¹H NMR (400 MHz, DMSO-d₆) δ 1.48-1.62 (m, 3H), 1.80-1.91 (m, 1H), 3.03-3.07 (m, 2H), 3.60 (t, J=6.4 Hz, 1H), 3.80 (s, 3H), 3.87 (s, 3H), 6.54-6.59 (m, 2H), 7.04 (brs, 2H), 7.75 (d, J=8.4 Hz, 1H), 7.90 (brs, 2H), 8.44 (s, 1H), 9.50 (brs, 1H); ESI-MS: m/z 323.30 (M+H)⁺.

### Example-14: Preparation of (E)-N²-{[2-hydroxy-5-(trifluoromethoxy)phenyl]methylidene}-L-arginine

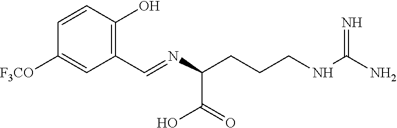 [SMILES: N=C(N)NCCC[C@H](/N=C/c1cc(OC(F)(F)F)ccc1O)C(=O)O]

L-arginine (0.81 g) was added to the solution of 2-hydroxy-5-(trifluoromethoxy) benzaldehyde (1.0 g) in ethanol (20 ml) and the reaction mixture was stirred at RT for 16 h. The reaction mixture was concentrated under vacuum and then crystalized in ethanol and n-hexane mixture to give 1.21 g of (E)-N²-{[2-hydroxy-5-(trifluoromethoxy)phenyl]methylidene}-L-arginine as a solid. ¹H NMR (400 MHz, DMSO-d₆) δ 1.47-1.51 (m 2H), 1.74-1.80 (m, 1H), 1.91-1.96 (m, 1H), 3.00-3.11 (m, 2H), 3.85 (t, J=6.4 Hz, 1H), 6.83 (d, J=9.2 Hz, 1H), 7.26-7.28 (m, 1H), 7.43 (s, 1H), 7.80 (brs, 4H), 8.48 (s, 1H), 9.48 (brs, 1H), 14.56 (brs, 1H); ESI-MS: m/z 363.30 (M+H)⁺.

### Example-15: Preparation of (E)-N²-[(3-chloro-5-fluoro-2-hydroxyphenyl) methylidene]-L-arginine

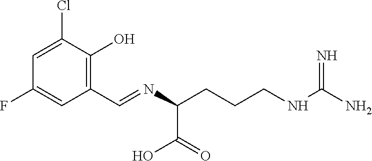 [SMILES: N=C(N)NCCC[C@H](/N=C/c1cc(F)cc(Cl)c1O)C(=O)O]

L-arginine (0.95 g) was added to the solution of 3-chloro-5-fluoro-2-hydroxy benzaldehyde (1.0 g) in ethanol (25 ml) and the reaction mixture was stirred at RT for 16 h. The reaction mixture was filtered and washed with n-hexane (25 ml) and dried under high vacuum t to give 1.37 g of (E)-N²-[(3-chloro-5-fluoro-2-hydroxyphenyl)methylidene]-L-arginine as a solid. ¹H NMR (400 MHz, DMSO-d₆) δ 1.48-1.55 (m 2H), 1.79-1.94 (m, 2H), 3.10-3.15 (m, 2H), 4.00 (t, J=6.4 Hz, 1H), 7.12 (dd, J=3.2, 5.6 Hz, 1H), 7.49 (dd, J=3.2, 5.2 Hz, 1H), 7.64 (brs, 4H), 8.45 (s, 1H), 9.31 (brs, 1H), 14.50 (brs, 1H); ESI-MS: m/z 331.60 (M+H)⁺.

### Example-16: Preparation of (E)-N²-[(4-methoxyphenyl)methylidene]-L-arginine

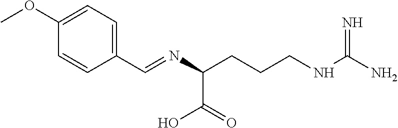 [SMILES: COc1ccc(/C=N/[C@@H](CCCNC(=N)N)C(=O)O)cc1]

Anisaldehyde (7.82 g) was added to the solution of L-arginine (10 g) in water (80 ml) and the mixture was stirred at RT for 7 h. The solid was filtered, washed with water (50 ml) and dried under high vacuum to give 16.6 g of (E)-N²-[(4-methoxyphenyl)methylidene]-L-arginine as a solid. ¹H NMR (400 MHz, DMSO-d₆) δ 1.40-1.54 (m, 2H), 1.64-1.69 (m, 1H), 1.85-1.90 (m, 1H), 3.08-3.09 (m, 2H), 3.65 (t, J=6.0 Hz, 1H), 3.77 (s, 3H), 6.95 (d, J=8.4 Hz, 2H), 7.64 (d, J=8.4 Hz, 2H), 7.40-8.15 (br s, 4H), 8.16 (s, 1H), 9.25 (br s, 1H); ESI-MS: m/z 293.20 (M+H)⁺.

### Example-17: Preparation of (E)-N²-[(3-hydroxy-4-methoxyphenyl)methylidene]-L-arginine

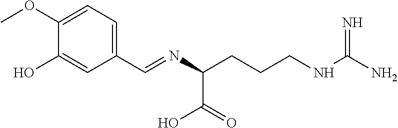 [SMILES: COc1ccc(/C=N/[C@@H](CCCNC(=N)N)C(=O)O)cc1O]

L-arginine (1.09 g) was added to the solution of iso-vanillin (1.0 g) in ethanol (25 ml) and the reaction mixture was stirred at RT for 5 h. The solid was filtered, washed with n-hexane (50 ml) and dried under high vacuum to give 0.86 g of (E)-N²-[(3-hydroxy-4-methoxyphenyl)methylidene]-L-arginine as a solid. ¹H NMR (400 MHz, DMSO-d₆) δ 1.40-1.52 (m, 2H), 1.62-1.40 (m, 1H), 1.83-1.91 (m, 1H), 3.02-3.06 (m, 2H), 3.62 (t, J=6.4 Hz, 1H), 3.44 (brs, 1H), 3.86 (s, 3H), 6.92 (d, J=8.4 Hz, 1H), 7.02 (d, J=9.6 Hz, 1H), 7.23 (d, J=1.2 Hz, 1H), 7.60 (brs, 5H), 8.06 (s, 1H); ESI-MS: m/z 309.10 (M+H)⁺.

### Example-18: Preparation of (E)-N²-[(3-chloro-4-methoxyphenyl)methylidene]-L-arginine

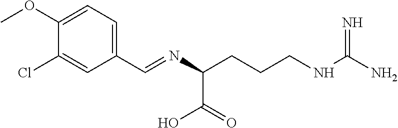 [SMILES: COc1ccc(/C=N/[C@@H](CCCNC(=N)N)C(=O)O)cc1Cl]

3-Chloro-p-anisaldehyde (1.5 g) was added to the slurry of L-arginine (1.45 g) in ethanol (30 ml) and the mixture was stirred at RT for 16 h. n-Hexane (60 ml) was added to the reaction mixture and it was stirred further 1 hr. The solid was filtered, washed with n-hexane (30 ml) and dried under high vacuum to give 1.68 g of (E)-N²-[(3-Chloro-4-methoxyphenyl)methylidene]-L-arginine as a solid. ¹H NMR (400 MHz, DMSO-d₆) δ 1.41-1.51 (m, 2H), 1.66-1.69 (m, 1H), 1.85-1.88 (m, 1H), 3.01-3.10 (m, 2H), 3.66 (t, J=6.4 Hz, 1H), 3.89 (s, 3H), 7.18 (d, J=8.4 Hz, 1H), 7.56 (brs, 2H), 7.63 (dd, J=1.6, 6.8, 1H), 7.77 (d, J=1.6 Hz, 1H), 7.95 (brs, 2H), 8.16 (s, 1H), 9.32 (brs. 1H); ESI-MS: m/z 349.60 (M+Na)+.

### Example-19: Preparation of (E)-N²-{[4-(methylsulfanyl)phenyl]methylidene}-L-arginine

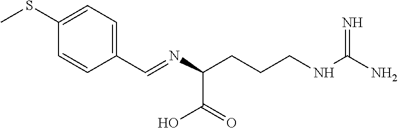 [SMILES: CSc1ccc(/C=N/[C@@H](CCCNC(=N)N)C(=O)O)cc1]

L-arginine (1.09 g) was added to the solution of 4-(methylthio)benzaldehyde (1.0 g) in ethanol (40 ml) and the reaction mixture was stirred at RT for 4 h. The solid was filtered, washed with n-hexane (50 ml) and dried under high vacuum to give 1.69 g of (E)-N²-{[4-(methylsulfanyl)phenyl]methylidene}-L-arginine as a solid. ¹H NMR (400 MHz, DMSO-d₆) δ 1.47-1.50 (m, 2H), 1.62-1.69 (m, 1H), 1.84-1.89 (m, 1H), 3.07-3.10 (m, 2H), 3.20 (s, 3H), 3.67 (t, J=6.4 Hz, 1H), 7.28 (d, J=8.4 Hz, 2H), 7.45 (brs, 2H), 7.64 (d, J=8.4 Hz, 2H), 8.01 (brs, 2H), 8.19 (s, 1H), 9.55 (s, 1H); ESI-MS: m/z 313.10 (M+H)⁺.

### Example-20: Preparation of (E)-N²-{[2-hydroxy-3-methoxy-5-(prop-2-en-1-yl)phenyl]methylidene}-L-arginine

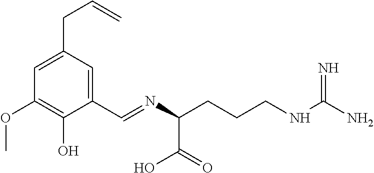 [SMILES: C=CCc1cc(/C=N/[C@@H](CCCNC(=N)N)C(=O)O)c(O)c(OC)c1]

L-arginine (0.86 g) was added to the solution of 2-hydroxy-3-methoxy-5-(prop-2-en-yl)benzaldehyde (1.0 g) in ethanol (25 ml) and the reaction mixture was stirred at RT for 16 h. The solid was filtered, washed with n-hexane (20 ml) and dried under high vacuum to give 1.35 g of (E)-N²-{[2-hydroxy-3-methoxy-5-(prop-2-en-1-yl)phenyl]methylidene}-L-arginine as a solid. ¹H NMR (400 MHz, DMSO-d₆) δ 1.47-1.50 (m, 2H), 1.70-1.90 (m, 2H), 3.10-3.19 (m, 2H), 3.24 (d, J=6.4 Hz, 1H), 3.42-3.45 (m, 1H), 3.73 (s, 3H), 3.81 (t, J=5.6 Hz, 1H), 5.08-5.08 (m, 2H), 5.89-5.99 (m, 1H), 6.88 (s, 1H), 6.72 (s, 1H), 7.53 (brs, 4H), 8.30 (s, 1H), 9.49 (brs, 1H), 14.14 (brs, 1H); ESI-MS: m/z 349.20 (M+H)⁺.

### Example-21: Preparation of (E)-N²-{[2,3-dimethoxy-5-(prop-2-en-1-yl)phenyl]methylidene}-L-arginine

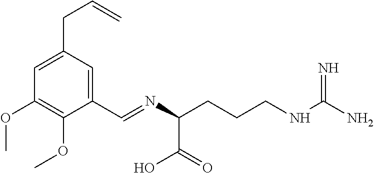 [SMILES: C=CCc1cc(/C=N/[C@@H](CCCNC(=N)N)C(=O)O)c(OC)c(OC)c1]

L-arginine (0.81 g) was added to the solution of 2,3-dimethoxy-5-(prop-2-en-yl)benzaldehyde (1.0 g) in ethanol (20 ml) and the reaction mixture was stirred at RT for 4 h. The solid was filtered, washed with n-hexane (50 ml) and dried under high vacuum to give 1.18 g of (E)-N²-{[2,3-dimethoxy-5-(prop-2-en-1-yl)phenyl]methylidene}-L-arginine as a solid. ¹H NMR (400 MHz, DMSO-d₆) δ 1.45-1.50 (m, 2H), 1.59-1.64 (m, 1H), 1.88-1.91 (m, 1H), 3.02-3.14 (m, 2H), 3.38 (d, J=6.8 Hz, 2H), 3.68 (t, J=6.8 Hz, 1H), 3.73 (s, 3H), 3.81 (s, 3H), 5.03-5.13 (m, 2H), 5.89-5.99 (m, 1H), 6.93 (d, J=2.0 Hz, 1H), 7.24 (d, J=2.0 Hz, 1H), 7.75 (brs, 4H), 8.45 (s, 1H), 9.57 (brs, 1H); ESI-MS: m/z 363.20 (M+H)⁺.

### Example-22: Preparation of (E)-N2-{[2-hydroxy-3-(prop-2-en-1-yl)phenyl]methylidene}-L-arginine

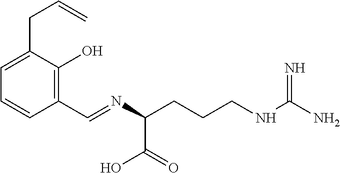 [SMILES: C=CCc1cccc(/C=N/[C@@H](CCCNC(=N)N)C(=O)O)c1O]

L-arginine (1.03 g) was added to the solution of 2-hydroxy-3-(prop-2-en-1-yl)benzaldehyde (1.0 g) in ethanol (10 ml) and the reaction mixture was stirred at RT for 16 h. The solid was filtered, washed with n-hexane (25 ml) and dried under high vacuum to give 1.64 g of (E)-N2-{[2-hydroxy-3-(prop-2-en-1-yl)phenyl]methylidene}-L-arginine as a solid. ¹H NMR (400 MHz, DMSO-d₆) δ 1.48-1.53 (m, 2H), 1.72-1.77 (m, 1H), 1.90-1.95 (m, 1H), 3.09-3.18 (m, 2H), 3.26-3.37 (m, 2H), 3.81 (t, J=5.6 Hz, 1H), 4.99-5.08 (m, 2H), 5.92-6.02 (m, 1H), 6.69 (t, J=7.6 Hz, 1H), 7.14 (d, J=0.8 Hz, 1H), 7.20 (d, J=0.8 Hz, 1H), 7.42 (brs, 4H), 8.41 (s, 1H), 9.54 (s, 1H), 14.62 (s, 1H); ESI-MS: m/z 319.5 (M+H)⁺.

### Example-23: Preparation of (E)-N²-[(1H-indol-3-yl)methylidene]-L-arginine

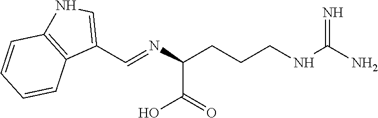 [SMILES: N=C(N)NCCC[C@H](/N=C/c1c[nH]c2ccccc12)C(=O)O]

Indole-3-carboxaldehyde (1.76 g) was added to the slurry of L-arginine (1.11 g) in ethanol (30 ml) and it was stirred at RT for 16 h. The solid was filtered, washed with n-hexane and dried under high vacuum to give to give 1.68 g of (E)-N²-[(1H-indol-3-yl)methylidene]-L-arginine as a solid. ¹H NMR (400 MHz, DMSO-d₆) δ 1.51-1.60 (m, 2H), 1.63-1.70 (m, 1H), 1.88-1.93 (m, 1H), 3.09-3.16 (m, 2H), 3.61 (t, J=6.4 Hz, 1H), 7.06 (t, J=7.2 Hz, 1H), 7.15 (t, J=7.2 Hz, 1H), 7.38 (d, J=8.0 Hz, 1H), 7.52 (br s. 2H), 7.71 (s, 1H), 8.01 (brs, 2H), 8.25 (d, J=8.0 Hz, 1H), 8.39 (s, 1H), 9.53 (brs, 1H), 11.46 (brs, 1H); ESI-MS: m/z 302.10 (M+H)⁺.

### Example-24: Preparation of (E)-N²-[(2H-1,3-benzodioxol-5-yl)methylidene]-L-arginine

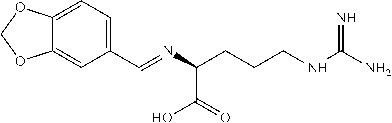 [SMILES: N=C(N)NCCC[C@H](/N=C/c1ccc2c(c1)OCO2)C(=O)O]

Piperonal (1 g) was added to the slurry of L-arginine (1.11 g) in ethanol (30 ml) and the mixture was stirred at RT for 16 h. n-Hexane (30 ml) was added to the reaction mixture and it was stirred for further 1 hr, The solid was filtered, washed with n-hexane and dried under high vacuum to give 0.91 g of (E)-N²-[(2H-1,3-benzodioxol-5-yl)methylidene]-L-arginine as a solid. ¹H NMR (400 MHz, DMSO-d₆) δ 1.40-1.54 (m, 2H), 1.61-1.66 (m, 1H), 1.85-1.87 (m, 1H), 3.07-3.09 (m 2H), 3.64 (t, J=6.4 Hz, 1H), 6.06 (d, J=4.4 Hz, 2H), 6.95 (d, J=8.0 Hz, 1H), 7.15 (d, J=11.2 Hz, 1H), 7.32 (s, 1H), 7.49 (brs, 2H), 7.56 (brs, 2H), 8.13 (s, 1H), 9.52 (brs, 1H); ESI-MS: m/z 307.30 (M+H)⁺.

### Example-25: Preparation of (E)-N²-[(furan-2-yl)methylidene]-L-arginine

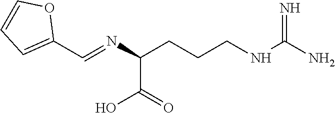 [SMILES: N=C(N)NCCC[C@H](/N=C/c1ccco1)C(=O)O]

Furfural (1.56 g) was added to the slurry of L-arginine (2 g) in ethanol (50 ml) and it was stirred at RT for 18 h under nitrogen atmosphere. The solvent was removed under vacuum and n-Hexane (20 ml) was added to the residue and it was stirred for 1 hr. The obtained solid was filtered, washed with n-hexane (10 ml) and dried under high vacuum to give 1.91 g (E)-N²-[(furan-2-yl)methylidene]-L-arginine as a solid. ¹H NMR (400 MHz, DMSO-d₆) δ 1.45-1.50 (m, 2H), 1.60-1.67 (m, 1H), 1.82-1.85 (m, 1H), 3.07-3.09 (m, 2H), 3.63 (t, J=6.80 Hz, 1H), 6.58-6.9 (m, 1H), 6.86 (d, J=3.2 Hz, 1H), 7.45 (brs, 2H), 7.55 (d, J=1.2 Hz, 1H), 8.06 (brs, 2H), 8.10 (s, 1H), 9.45 (brs, 1H); ESI-MS: m/z 253.20 (M+H)⁺.

### Example-26: Preparation of (E)-N²-[(5-methylfuran-2-yl)methylidene]-L-arginine

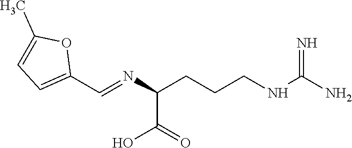 [SMILES: Cc1ccc(/C=N/[C@@H](CCCNC(=N)N)C(=O)O)o1]

L-arginine (1.5 g) was added to the solution of 5-methylfurfural (1.0 g) in ethanol (20 ml). The reaction mixture was stirred at RT for 16 h. The solid was filtered, washed with ethanol (10 ml) and dried under high vacuum to give 1.99 g of (E)-N²-[(5-methylfuran-2-yl)methylidene]-L-arginine as a solid. ¹H NMR (400 MHz, DMSO-d₆) δ 1.45-1.85 (m, 4H), 2.31 (s, 3H), 3.06-3.10 (m, 2H), 3.57 (t, J=6.4 Hz, 1H), 6.22 (d, J=2.8 Hz, 1H), 6.71 (d, J=2.8 Hz, 1H), 7.50 (brs, 5H), 7.99 (s, 1H); ESI-MS: m/z 267.20 (M)+.

### Example-27: Preparation of (E)-N²-{[5-(hydroxymethyl)furan-2-yl]methylidene}-L-arginine

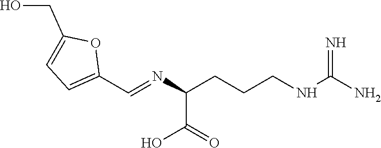 [SMILES: N=C(N)NCCC[C@H](/N=C/c1ccc(CO)o1)C(=O)O]

L-arginine (1.32 g) was added to the solution of 5-(hydroxymethyl)furan-2-carbaldehyde (1.0 g) in ethanol (25 ml). The reaction mixture was stirred at RT for 16 h. The solid was filtered, washed with ethanol (10 ml) and n-hexane (25 ml) and then dried under high vacuum to give 1.71 g of (E)-N²-{[5-(hydroxymethyl)furan-2-yl]methylidene}-L-arginine as a solid. ¹H NMR (400 MHz, DMSO-d₆) δ 1.40-1.55 (m, 2H), 1.60-1.67 (m, 1H), 1.81-1.90 (m, 1H), 3.04-3.12 (m, 2H), 3.61 (t, J=6.4 Hz, 1H), 4.42 (s, 2H), 5.50 (brs, 1H), 6.39 (d, J=2.8 Hz, 1H), 6.77 (d, J=2.4 Hz, 1H), 7.48 (brs, 4H), 8.09 (s, 1H), 9.50 (brs, 1H); ESI-MS: m/z 283.20 (M)+.

### Example-28: Preparation of (E)-N2-[(naphthalen-1-yl)methylidene]-L-arginine

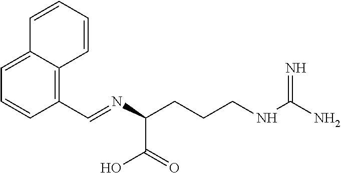 [SMILES: N=C(N)NCCC[C@H](/N=C/c1cccc2ccccc12)C(=O)O]

L-arginine (0.96 g) was added to the solution of 1-napthaldehyde (1 g) in ethanol (15 ml) at room temperature. The reaction mixture was stirred for 18 h at RT under nitrogen atmosphere. The solid product was filtered, washed with cold ethanol (10 ml) followed by n-hexane (40 ml) and then dried under high vacuum to give 1.19 g of (E)-N2-[(naphthalen-1-yl)methylidene]-L-arginine as a solid. ¹H NMR (400 MHz, DMSO-d₆) δ 1.56 (br s, 2H), 1.74-1.81 (m, 1H), 1.97-2.05 (m, 1H), 3.13-3.14 (m, 2H), 3.82 (t, J=6.4 Hz, 1H), 7.34 (br s, 4H), 7.55-7.63 (m, 3H), 7.88 (d, J=6.8 Hz, 1H), 7.99 (t, J=7.2 Hz, 2H) 8.86 (s, 1H), 9.17 (d, J=8.4 Hz, 1H), 9.79 (br s, 1H); ESI-MS: m/z 313.12 (M+H)⁺.

### Example-29: Preparation of (E)-N2-[(pyridin-3-yl)methylidene]-L-arginine

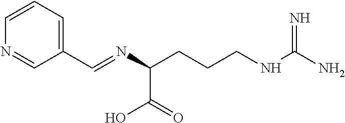 [SMILES: N=C(N)NCCC[C@H](/N=C/c1cccnc1)C(=O)O]

L-arginine (0.9 g) was added to the solution of nicotinaldehyde (1 g) in ethanol (10 ml) at room temperature. The reaction mixture was stirred for 18 h at RT under nitrogen atmosphere. The solid product was filtered, washed with cold ethanol (10 ml) followed by n-hexane (30 ml) and then dried under high vacuum to give 0.91 g of (E)-N2-[(pyridin-3-yl)methylidene]-L-arginine as a solid. ¹H NMR (400 MHz, DMSO-d₆) δ 1.46-1.52 (m, 2H), 1.68-1.73 (m, 1H), 1.90-1.93 (m, 1H), 3.09 (br s, 1H), 3.74 (t, J=6.4 Hz, 1H), 7.29 (br s, 5H), 7.45 (dd, J=5.0 Hz, 7.8 Hz, 1H), 8.11 (d, J=8.0 Hz, 1H), 8.33 (s, 1H), 8.61 (dd, J=1.6 Hz, 4.8 Hz, 1H), 8.86 (s, 1H), 9.71 (br s, 1H); ESI-MS: m/z 264.20 (M+H)⁺.

### Example-30: Preparation of (E)-N2-[(naphthalen-2-yl)methylidene]-L-arginine

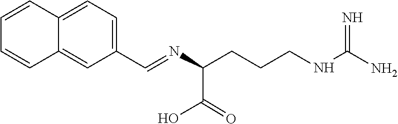 [SMILES: N=C(N)NCCC[C@H](/N=C/c1ccc2ccccc2c1)C(=O)O]

L-arginine (0.90 g) was added to the solution of 2-napthaldehyde (1 g) in ethanol (10 ml) at room temperature. The reaction mixture was stirred for 18 h at RT under nitrogen atmosphere. The solid product was filtered, washed with cold ethanol (10 ml) followed by n-hexane (40 ml) and then dried under high vacuum to give 1.13 g of (E)-N2-[(naphthalen-2-yl)methylidene]-L-arginine as a solid. ¹H NMR (400 MHz, DMSO-d₆) δ 1.49-1.556 (m, 2H), 1.71-1.76 (m, 1H), 1.94-1.97 (m, 1H), 3.11 (br s, 2H), 3.78 (t, J=6.4 Hz, 1H), 7.32 (br s, 4H), 7.54-7.58 (m, 2H), 7.92-7.96 (m, 3H), 7.98-8.01 (m, 1H) 8.17 (s, 1H), 8.42 (s, 1H), 9.80 (br s, 1H); ESI-MS: m/z 313.28 (M+H)⁺.

### Example-31: Preparation of (E)-N2-[(thiophen-3-yl)methylidene]-L-arginine

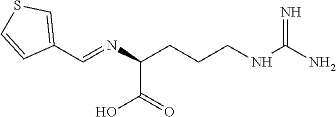 [SMILES: N=C(N)NCCC[C@H](/N=C/c1ccsc1)C(=O)O]

L-arginine (0.9 g) was added to the solution of thiophene-3-carbaldehyde (1 g) in ethanol (10 ml) at room temperature. The reaction mixture was stirred for 18 h at RT under nitrogen atmosphere. The solid product was filtered, washed with cold ethanol (10 ml) followed by n-hexane (20 ml) and then dried under high vacuum to give 0.48 g of (E)-N2-[(thiophen-3-yl)methylidene]-L-arginine as a solid. ¹H NMR (400 MHz, DMSO-d₆) δ 1.47 (br s, 2H), 1.61-1.66 (m, 1H), 1.84-1.88 (m, 1H), 3.06-3.09 (m, 2H), 3.62 (t, J=6.4 Hz, 1H), 7.33-7.60 (br s, 4H), 7.43 (d, J=4.4 Hz, 1H), 7.55-7.56 (m, 1H), 7.87 (s, 1H) 8.25 (s, 1H), 9.69 (br s, 1H); ESI-MS: m/z 269.40 (M+H)⁺.

### Example-32: Preparation of (E)-N2-{[2-methoxy-3-(prop-2-en-1-yl)phenyl]methylidene}-L-arginine

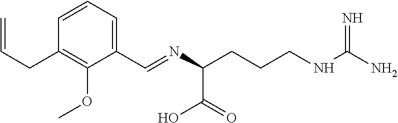 [SMILES: C=CCc1cccc(/C=N/[C@@H](CCCNC(=N)N)C(=O)O)c1OC]

L-arginine (1.89 g) was added to the solution of 2-methoxy-3-(prop-2-en-1-yl) benzaldehyde (2 g) in isopropyl alcohol (20 ml) at room temperature. The reaction mixture was stirred for 18 h at RT under nitrogen atmosphere. The solid product was filtered, washed with n-hexane (20 ml) and then dried under high vacuum to give 2.34 g of (E)-N2-{[2-methoxy-3-(prop-2-en-1-1)phenyl]methylidene}-L-arginine as a solid. ¹H NMR (400 MHz, DMSO-d₆) δ 1.50 (br s, 2H), 1.66-1.71 (m, 1H), 1.88-1.92 (m, 1H), 3.09 (d, J=6.4 Hz, 2H) 3.40 (d, J=6.4 Hz, 2H), 3.72 (s, 3H), 3.74-3.76 (m, 1H), 5.05-509 (m, 2H), 5.93-6.01 (m, 1H), 7.12 (t, J=7.6 Hz, 1H), 7.26 (d, J=7.6 Hz, 1H), 7.31-7.36 (br s, 4H), 7.73 (d, J=8.0 Hz, 1H), 8.48 (s, 1H), 9.70 (br s, 1H); ESI-MS: m/z 333.20 (M+H)⁺.

### Example-33: Preparation of (E)-N2-[(7-methoxy-2H-1,3-benzodioxol-5-yl)methylidene]-L-arginine

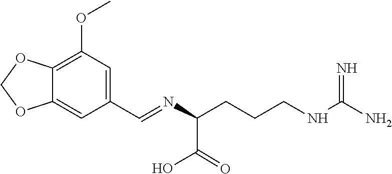 [SMILES: COc1cc(/C=N/[C@@H](CCCNC(=N)N)C(=O)O)cc2c1OCO2]

L-arginine (0.96 g) was added to the solution of 7-methoxy-2H-1,3-benzodioxole-5-carbaldehyde (1 g) in ethanol (20 ml) at room temperature. The reaction mixture was stirred for 18 h at RT under nitrogen atmosphere. The solid product was filtered, washed with n-hexane (20 ml) and then dried under high vacuum to give 1.23 g of (E)-N2-[(7-methoxy-2H-1,3-benzodioxol-5-yl)methylidene]-L-arginine as a solid. ¹H NMR (400 MHz, DMSO-d₆) δ 1.46 (br s, 2H), 1.63-1.65 (m, 1H), 1.87-1.88 (m, 1H), 3.05-3.37 (m, 2H), 3.65 (t, J=6.6 Hz, 1H), 3.85 (s, 3H), 6.05 (s, 2H), 6.95 (s, 1H), 7.04 (s, 1H), 7.46 (br s, 3H), 7.99 (br s, 1H), 8.12 (s, 1H), 9.48 (br s, 1H); ESI-MS: m/z 337.20 (M+H)⁺.

### Example-34: Preparation of (E)-N2-[(2H-1,3-benzodioxol-4-yl)methylidene]-L-arginine

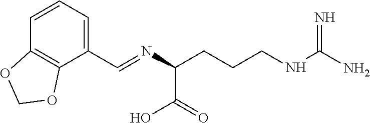 [SMILES: N=C(N)NCCC[C@H](/N=C/c1cccc2c1OCO2)C(=O)O]

L-arginine (1.11 g) was added to the solution of 2H-1,3-benzodioxole-4-carbaldehyde in ethanol (20 ml) at room temperature. The reaction mixture was stirred for 18 h at RT under nitrogen atmosphere. The solid product was filtered, washed with n-hexane (20 ml) and then dried under high vacuum to give 1.41 g of (E)-N2-[(2H-1,3-benzodioxol-4-yl)methylidene]-L-arginine as a solid. ¹H NMR (400 MHz, DMSO-d₆) δ 1.47 (br s, 2H), 1.63-1.72 (m, 1H), 1.84-1.89 (m, 1H), 3.07-3.09 (m, 2H), 3.69 (t, J=6.4 Hz, 1H), 6.09 (s, 2H), 6.85 (t, J=7.6 Hz, 1H), 6.95 (d, J=8.0 Hz, 1H), 7.26 (d, J=7.6 Hz, 1H), 7.52-7.91 (br s, 5H), 8.32 (s, 1H), 9.36 (br s, 1H); ESI-MS: m/z 307.20 (M+H)⁺.

### Example-35: Stability Studies of the Compounds of the Present Invention

The compounds of present invention were stored under conditions as shown in below table [room temperature and on accelerated storage condition (ATS)] and the purity was estimated by HPLC. The properly packed material was subjected to the conditions mentioned in the table below.

TABLE 1

| | Compound | Day Zero | | Day 14 | |
|---|---|---|---|---|---|
| | of the | Condition | | Condition | |
| Sr. | present | 25° C. ± | 54° C. ± | 25° C. ± | 54° C. ± |
| No. | invention | 2° C. | 2° C. | 2° C. | 2° C. |
| 1 | Example 2 | 99.71 | 99.60 | 99.70 | 99.55 |
| 2 | Example 11 | 99.10 | 99.20 | 99.00 | 98.85 |
| 3 | Example 22 | 99.60 | 99.63 | 99.50 | 99.30 |
| 4 | Example 24 | 98.90 | 98.75 | 98.80 | 98.90 |
| 5 | Example 26 | 99.20 | 99.00 | 99.10 | 99.00 |
| 6 | Example 30 | 99.70 | 99.65 | 99.65 | 99.45 |

The following examples illustrate the basic methodology and versatility of the composition of the invention. It should be noted that this invention is not limited to these exemplifications. The form of the composition, excipients and the concentrations of actives and excipients can be replaced by any other forms, excipients and concentrations as covered in the present invention.

### Example-36: General Procedure for the Preparation of Water Disintegrable Granules (GR)

The compound of the present invention is ground to desired particle below 50 microns in jet mill, then all the ingredients are added and mixed in ribbon blender to obtain homogeneous powder. Required quantity of water is added to the above mixture to prepare dough and the material is then granulated in suitable equipment and dried to obtain granules.

The following Water Disintegrable Granular (GR) compositions were prepared as per Example 36.

TABLE 2
Formulation (parts by weight)

| Composition No. | I | II | III | IV | V | VI |
|---|---|---|---|---|---|---|
| Technical | Ex. 11 | Ex. 15 | Ex. 26 | Ex. 22 | Ex. 31 | Ex. 24 |
| | 92 | 90.8 | 91.5 | 80.5 | 90.6 | 84.2 |
| Sodium lauryl | 2 | 2 | 1.5 | — | — | — |
| sulphate | | | | | | |
| Sodium ligno | — | — | — | 2 | — | — |
| sulphonate | | | | | | |
| Sodium isopropyl | — | — | — | — | 2 | — |
| naphthalene sulfonate | | | | | | |
| Sodium naphthalene | — | — | — | — | — | 2 |
| sulfonate condensate | | | | | | |
| PEG 400 | — | — | — | 1.5 | — | — |
| Alcohol ethoxylate | — | — | — | — | 0.5 | — |
| Sodium sulphate | — | — | — | 5 | — | — |
| Bentonite | 2.8 | — | — | 9 | 4.9 | 6.8 |
| Talc | 2.7 | — | — | — | 1 | 3 |
| Corn starch | 0.5 | 2 | 2 | — | — | — |
| Perlite | — | 2.2 | 4 | — | — | 4 |
| Mica | — | 3 | — | — | — | — |
| Silica | — | — | 1 | 2 | — | — |
| Titanium oxide | — | — | — | — | 1 | — |

### Example-37: General Procedure for the Preparation of Extruded Granules (WG)

The compound of the present invention is blended with dispersing agent, wetting agents and inert excipients in a ribbon blender and then ground to desired particle size in jet mill. Required quantity of water is added to the above mixture to prepare dough and the material is then granulated in suitable equipment and dried to obtain granules.

The following Extruded Granular (WG) compositions were prepared as per Example 37.

TABLE 3
Formulation (parts by weight)

| Composition No. | I | II | III | IV | V | VI |
|---|---|---|---|---|---|---|
| Technical | Ex. 1 | Ex. 15 | Ex. 22 | Ex. 24 | Ex. 31 | Ex. 26 |
| | 25.2 | 25.2 | 36.8 | 41.2 | 72.2 | 30.7 |
| Sodium alkyl | 11.5 | — | — | — | — | — |
| naphthalene | | | | | | |
| sulfonate | | | | | | |
| Sodium naphthalene | — | — | — | 6 | — | — |
| sulfonate condensate | | | | | | |
| Sodium sulphate | 10 | 13.3 | — | 10 | — | 19 |
| Sodium ligno | — | — | 7 | — | 15 | 8 |
| sulphonate | | | | | | |
| Polycarboxylate | 2 | 2 | — | — | | 2 |
| Sodium lauryl | 1.5 | — | — | — | 2.5 | 3 |
| sulphate | | | | | | |
| Ethoxylated | — | 1.5 | — | — | — | — |
| polyarylphenol | | | | | | |
| phosphate amine salt | | | | | | |
| Polyalkoxylated | — | — | — | 1.5 | — | — |
| butyl ether | | | | | | |
| Dioctyl Sodium | — | — | 3 | — | — | — |
| Sulfosuccinate | | | | | | |
| Tristyryl phenol | — | — | — | — | 2.5 | — |
| phosphate | | | | | | |
| Silicon defoamer | — | — | — | — | 0.2 | — |
| lactose | — | — | 15 | — | 7.6 | 15 |
| Sodium citrate | — | — | 2 | — | — | — |
| Maltodextrin | 17 | 18 | — | — | — | — |
| Clay | — | — | 6.2 | 18.8 | — | 22.3 |
| Silica | — | — | — | 2.5 | — | — |
| Soapstone clay | — | — | 28 | — | — | — |
| Perlite | 22.8 | 30 | — | 20 | — | — |
| Aluminium silicate | 10 | 10 | — | — | — | — |
| Mica | — | — | 2 | — | — | — |

### Example-38: General Procedure for the Preparation of Spray Dried Granules (WG)

The compound of the present invention is blended with all the ingredients in 100 parts of water and milled to desired particle size. The milled slurry is then spray dried/fluid bed dried to obtain granules.

The following spray dried granular (WG) compositions were prepared as per Example 38.

TABLE 4
Formulation (parts by weight)

| Composition No. | I | II | III | IV |
|---|---|---|---|---|
| Technical | Ex. 15 | Ex. 31 | Ex. 11 | Ex. 22 |
| | 73.8 | 67 | 77 | 60.5 |
| Sodium alkyl naphthalene | 2 | — | — | — |
| sulfonate | | | | |
| Sodium ligno sulphonate | 12 | 10 | 9 | |
| Modified ligno sulphonate | | — | — | 8 |
| Sodium sulphate | 11.2 | — | — | |
| Sodium naphthalene sulfonate | — | 2 | — | 5 |
| condensate | | | | |
| Naphthalene Sulphonic Acid and | 1 | — | — | — |
| Phenol Sulphonic Acid | | | | |
| condensation | | | | |
| 4-4 dihydroxydiphenyl | — | — | 3 | — |
| sulphonate condensate | | | | |
| Ethoxylated polyarylphenol | — | 3 | — | — |
| phosphate amine salt | | | | |
| Polyvinyl alcohol (PVA) | — | — | 0.2 | 0.2 |
| Sodium hexametaphosphate | — | — | — | 2 |
| Sodium tripolyphsophate | — | — | 1 | — |
| Polycarboxylate | — | 2.2 | — | — |
| Sodium citrate | — | — | — | 4.3 |
| modified starch | — | 3.2 | — | — |
| Clay | — | — | 9.8 | — |
| Kaolin clay | — | — | — | 20 |
| Fumed silica | — | 12.6 | — | — |

### Example-39: General Procedure for the Preparation of WP

The compound of the present invention is blended with required quantity of dispersing agent, wetting agents and inert ingredients in a ribbon blender. Blended material is passed through jet mill to reduce the particle size and further blended for one hour to obtain product of desired composition.

The following Wettable Powder (WP) compositions were prepared as per Example 39.

TABLE 5
Formulation (parts by weight)

| Composition No. | I | II | III | IV | V | VI |
|---|---|---|---|---|---|---|
| Technical | Ex. 11 | Ex. 15 | Ex. 25 | Ex. 26 | Ex. 22 | Ex. 31 |
| | 86.8 | 81.4 | 82 | 80.6 | 70.6 | 72.2 |
| Sodium alkyl | 3 | — | — | — | 2.5 | — |
| naphthalene | | | | | | |
| sulfonate | | | | | | |
| Sodium isopropyl | — | 3 | — | — | — | — |
| napthalene sulfonate | | | | | | |
| Sodium butyl | — | — | — | 3 | — | — |
| naphthalene | | | | | | |
| sulfonate | | | | | | |
| Sodium ligno | 7 | 4 | 7.8 | 7 | — | 8 |
| sulphonate | | | | | | |
| Sodium naphthalene | — | — | — | — | 4 | 6 |
| sulfonate condensate | | | | | | |
| blend of alkyl | — | — | — | — | — | 4 |
| naphthalene | | | | | | |
| sulfonate | | | | | | |
| Dioctyl Sodium | — | — | 3 | — | — | — |
| Sulfosuccinate | | | | | | |
| Polycarboxylate | — | 5 | — | — | — | — |
| Sodium lauryl | — | — | — | 2 | — | — |
| sulphate | | | | | | |
| Clay | — | 6.6 | — | — | — | — |
| Silica | 3.2 | — | 2 | 3 | 3 | 4 |
| Kaolin clay | — | — | 5.2 | 4.4 | 19.9 | 5.8 |

### Example-40: General Procedure for the Preparation of Suspension Concentrate

Required quantity of surfactant and anti-freezing agent is mixed with defined quantity of water. The compound of the present invention is further added to the homogenized blend and it is stirred continuously for approximately 20 minutes until the total mixture is homogeneous. Half quantity of defoamer is added under continuous homogenization to obtain liquid suspension. Subsequently, the suspension obtained is passed through the wet mill to reduce the particle size. The required qty of thickener, preservative and balance water and defoamer is added to get the product of desired composition.

### Example-41: General Procedure for the Preparation of Coated Granules (GR)

Required quantity of stabilizer, surfactant, coloring agent, binder is blended and heated to desired temperature in drum coater Required quantity of river sand is charged into the drum coater and mixed. The compound of the present invention of desired particle size is charged into the ribbon blender and mixed to obtain product of desired composition.

### Example-42: General Procedure for the Preparation of Powder for Dry Seed Treatment (DS)

The compound of the present invention is blended with required quantity of pigment, wetting agent and inert ingredients in a ribbon blender. Blended material is passed through jet mill to reduce the particle size and further blended for one hour to obtain product of desired composition.

### Example-43: General Procedure for the Preparation of Water Dispersible Powder for Slurry Treatment (WS)

The compound of the present invention is blended with required quantity of pigment, dispersing agent, wetting agents and inert ingredients in a ribbon blender. Blended material is passed through jet mill to reduce the particle size and further blended for one hour to obtain product of desired composition.

The following Suspension concentrate (SC), coated granules (GR), Powder for dry seed treatment (DS) and Water dispersible powder for slurry treatment (WS) compositions were prepared as per Examples 40, 41, 42 and 43 respectively.

TABLE 6

| | | | | | | | Water | |
|---|---|---|---|---|---|---|---|---|
| | | | | | | | dispersible | |
| | | | | | Powder for | | powder for | |
| | Suspension | | Coated | | dry seed | | slurry | |
| Formulation | concentrate | | granules | | treatment | | treatment | |
| (parts by weight) | (SC) | | (GR) | | (DS) | | (WS) | |
| Technical | Ex. 31 | Ex. 11 | Ex. 25 | Ex. 15 | Ex. 26 | Ex. 22 | Ex. 1 | Ex. 24 |
| | 10.6 | 15.2 | 4.5 | 20 | 52.8 | 40.4 | 52.8 | 30.3 |
| Isodecyl alcohol | — | — | 3 | 2 | — | — | — | — |
| polyglycolether | | | | | | | | |
| Polyvinyl | — | — | 0.5 | — | — | — | — | — |
| alcohol (PVA) | | | | | | | | |
| 1,2- | 0.1 | 0.5 | — | — | — | — | — | — |
| benzisothiazolin- | | | | | | | | |
| 3-one | | | | | | | | |
| Anionic | 3.5 | 4.5 | — | — | — | — | — | — |
| tristyrylphenol | | | | | | | | |
| phosphate | | | | | | | | |
| Sodium | 2 | — | — | — | — | 3 | 3 | 4 |
| naphthalene | | | | | | | | |
| sulfonate | | | | | | | | |
| condensate | | | | | | | | |
| Sodium alkyl | — | 2.5 | — | — | 3.5 | — | 2 | — |
| naphthalene | | | | | | | | |
| sulfonate | | | | | | | | |
| Sodium ligno | — | — | — | — | 1 | 3 | 8 | 4 |
| sulphonate | | | | | | | | |
| Silicon defoamer | 0.5 | 0.5 | — | — | — | — | — | — |
| Propylene glycol | 5 | 5 | — | 2 | — | — | — | — |
| Xantham gum | 0.12 | 0.12 | — | | — | — | — | — |
| River sand | — | — | 91.2 | 75.2 | — | — | — | — |
| Silica | — | — | — | — | 2 | — | 2 | — |
| Clay | — | — | — | — | 36.7 | 50.6 | 29.2 | 57.7 |
| Pigment | — | — | — | — | 4 | 3 | 3 | 4 |
| Dye | — | — | 0.8 | 0.8 | — | — | — | — |
| Water | 78.18 | 71.68 | — | — | — | — | — | — |

### Example-43: Determination of the Nematocidal Activity Against Root-Knot Nematodes (Meloidogyne Spp)

The compound to be tested (4 mg) was dissolved in 40 μl of DMSO to make 100,000 PPM concentration stock solution. The clear solution was obtained by vortexing. The nematode suspension (250 μl) having 70-100 juveniles (J2) of Root-knot nematodes Meloidogyne spp. pipetted out to each cell of 24 well bioassay plate. Sterile water (1 ml) was pipetted out in fresh 2.5 ml Eppendorf micro-centrifuge tubes. The desired concentration (500 ppm) of the compound was prepared by adding stock solution in 2.5 ml Eppendorf micro-centrifuge tubes. For each treatment 4 replications were maintained. The bioassay plates were incubated at 22±2° C. and 70±5% RH in humidity chambers. The Dead/inactive nematodes were counted under microscope at 24, 48, 72 and 96 hours after incubation and the percent mortality is calculated.

TABLE 7
Rating Table

| % Mortality | Rating |
|---|---|
| 70-100 | A |
| 50-69 | B |
| 0 to 49 | C |

TABLE 8
Biological results

| Example No. | Rating |
|---|---|
| 1 | A |
| 2 | A |
| 3 | B |
| 4 | B |
| 5 | A |
| 6 | A |
| 7 | A |
| 8 | A |
| 9 | C |
| 10 | B |
| 11 | A |
| 12 | C |
| 13 | C |
| 14 | B |
| 15 | C |
| 16 | A |
| 17 | C |
| 18 | B |
| 19 | B |
| 20 | A |
| 21 | A |
| 22 | A |
| 23 | B |
| 24 | B |
| 25 | A |
| 26 | A |
| 27 | B |
| 28 | B |
| 29 | B |
| 30 | A |
| 31 | B |
| 32 | B |
| 33 | B |
| 34 | B |
| — | — |
| — | — |

1-22. (canceled)

23. A compound of formula (I) 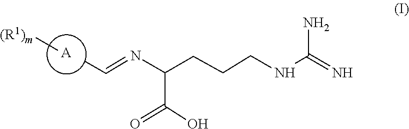
or an agrochemically acceptable salt, stereoisomer, enantiomer, tautomer, or N-oxide thereof, wherein,

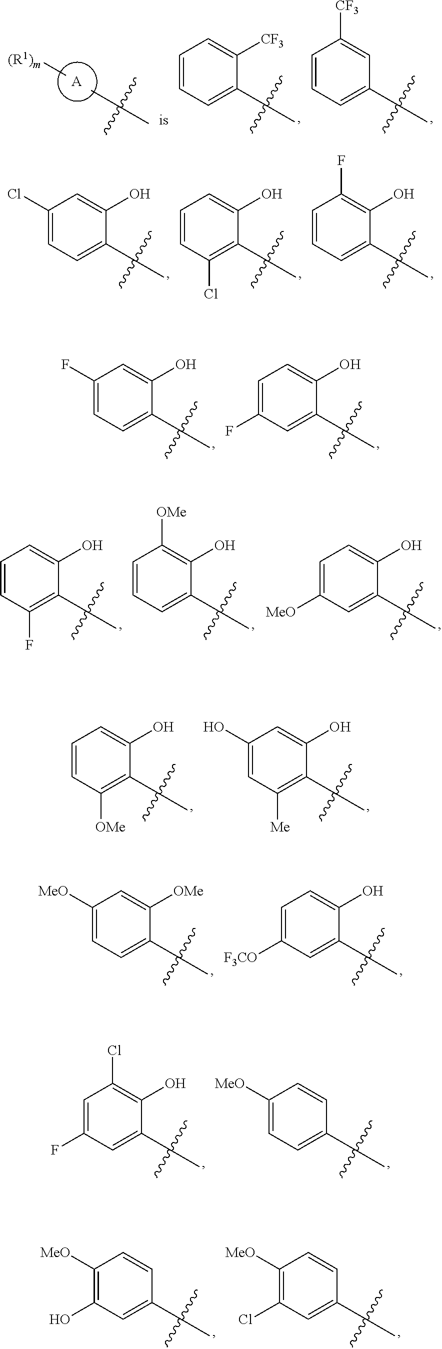

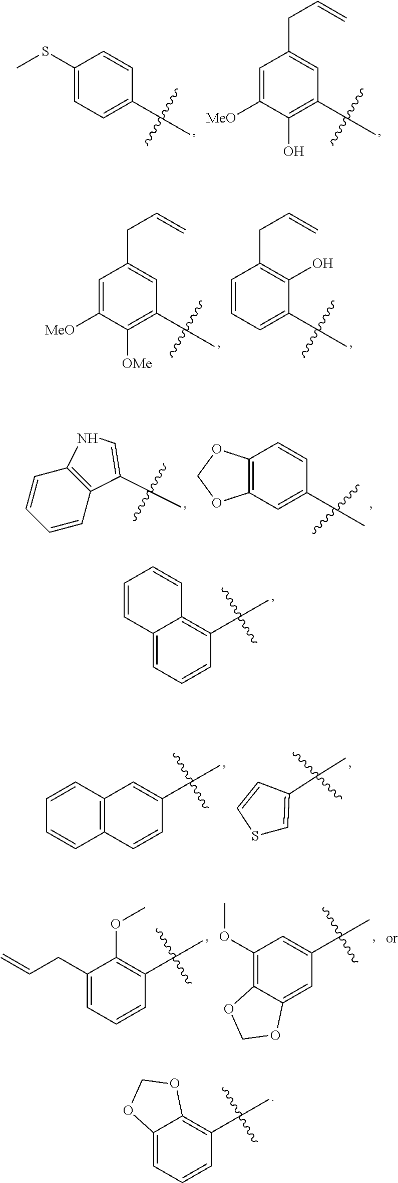

24. The compound according to claim 23, wherein

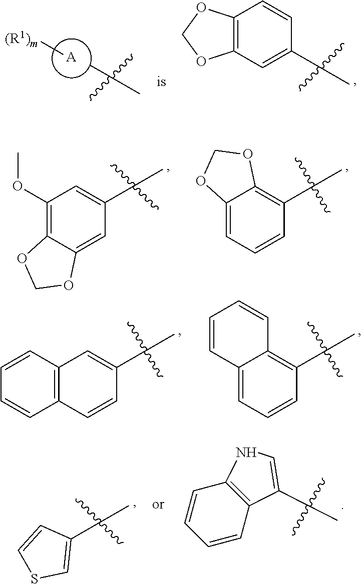

25. The compound according to claim 23, wherein the compound is selected from 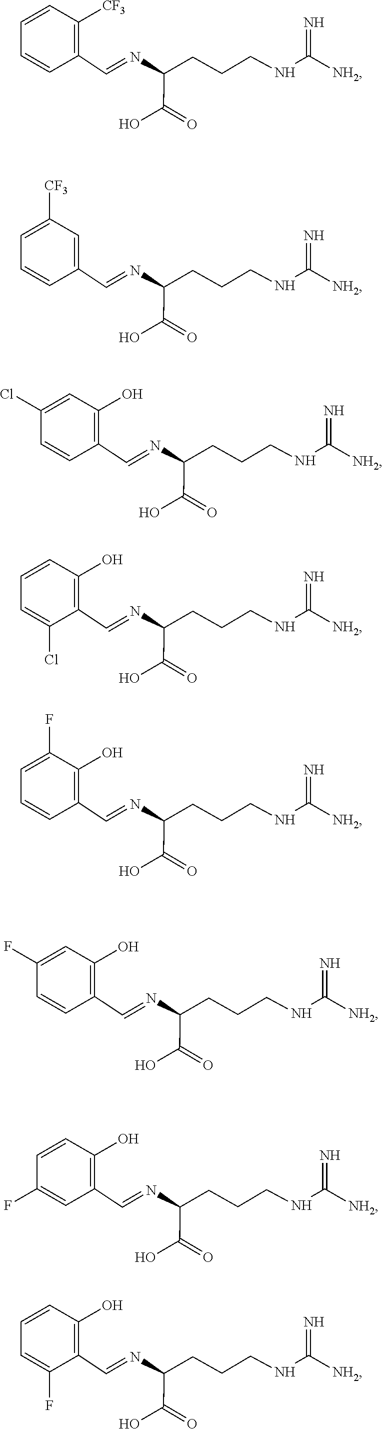 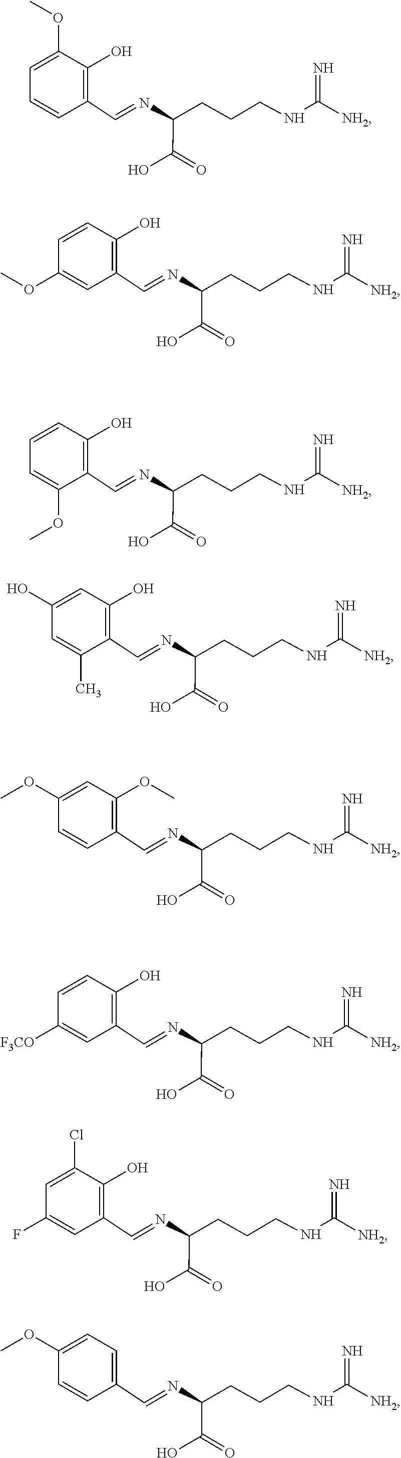 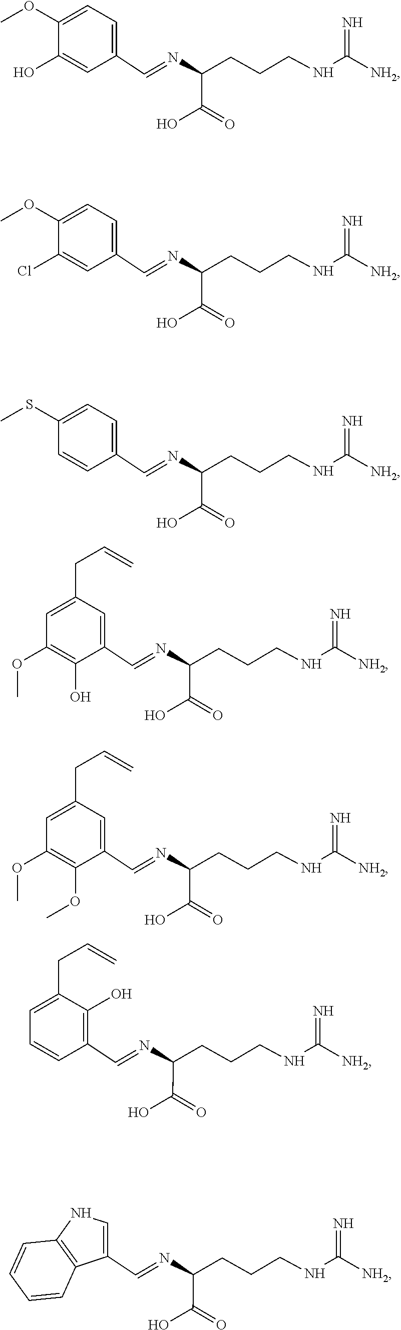 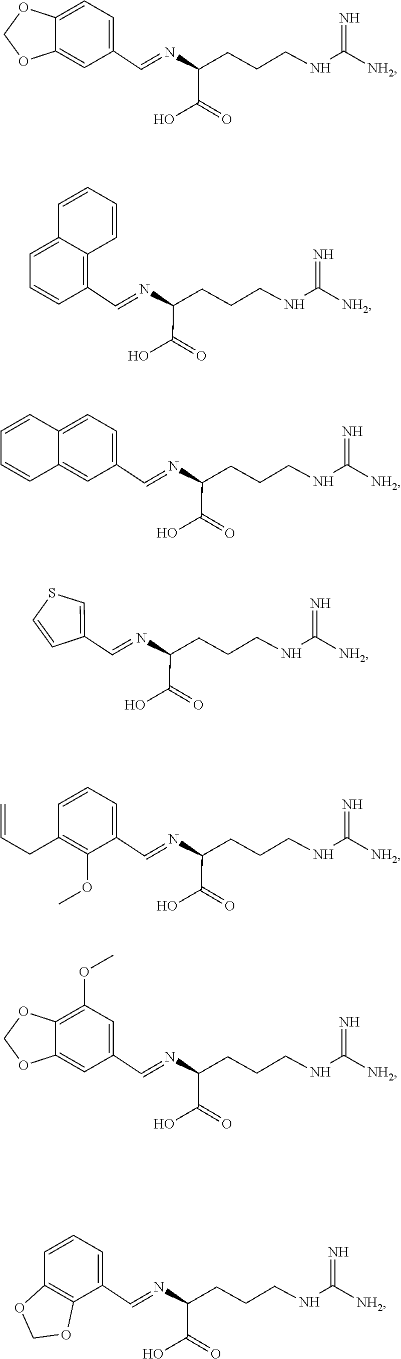
or agrochemically acceptable salts or N-oxides thereof.

26. The compound of according to claim 23, wherein the compound is selected from 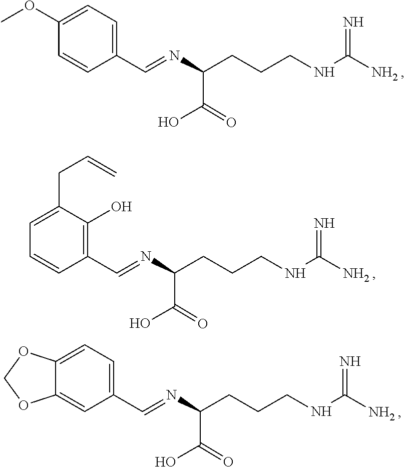 [SMILES: COc1ccc(/C=N/[C@@H](CCCNC(=N)N)C(=O)O)cc1 ; C=CCc1cccc(/C=N/[C@@H](CCCNC(=N)N)C(=O)O)c1O ; N=C(N)NCCC[C@H](/N=C/c1ccc2c(c1)OCO2)C(=O)O]
or agrochemically acceptable salts or N-oxides thereof.

27. The compound of according to claim 23, wherein the compound is 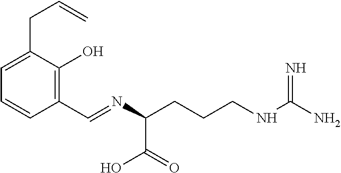 [SMILES: C=CCc1cccc(/C=N/[C@@H](CCCNC(=N)N)C(=O)O)c1O]
or an agrochemically acceptable salt or N-oxide thereof.

28. An agricultural composition comprising a compound of claim 23 and at least one agrochemically acceptable excipient.

29. The agricultural composition according to claim 28, wherein the agrochemically acceptable excipient is selected from one or more of surfactants, disintegrating agents, fillers or carriers or diluents, spreading agents, colorants, anticaking agents, binders, buffers or pH adjusters or neutralizing agents, pigments, stabilizers, antifoaming agents or defoamers, penetrants, structuring agents, humectants, sticking agents, anti-freezing agent or freeze point depressants, chelating or complexing or sequestering agents, or preservatives.

30. The agricultural composition according to claim 28, wherein the compound is selected from 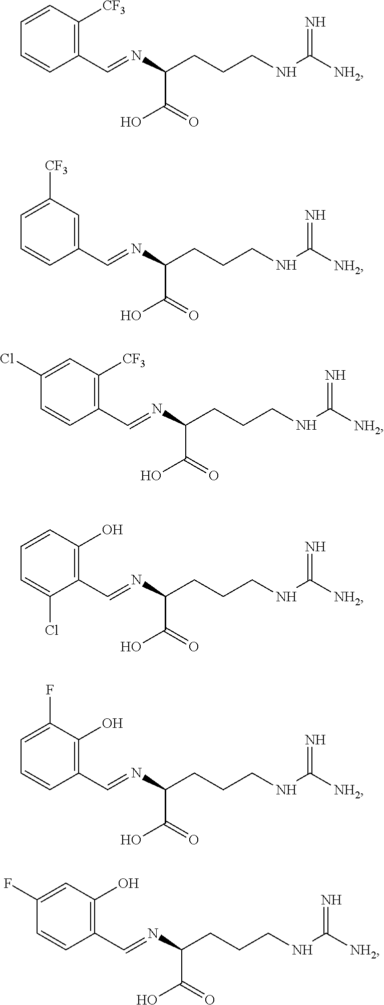 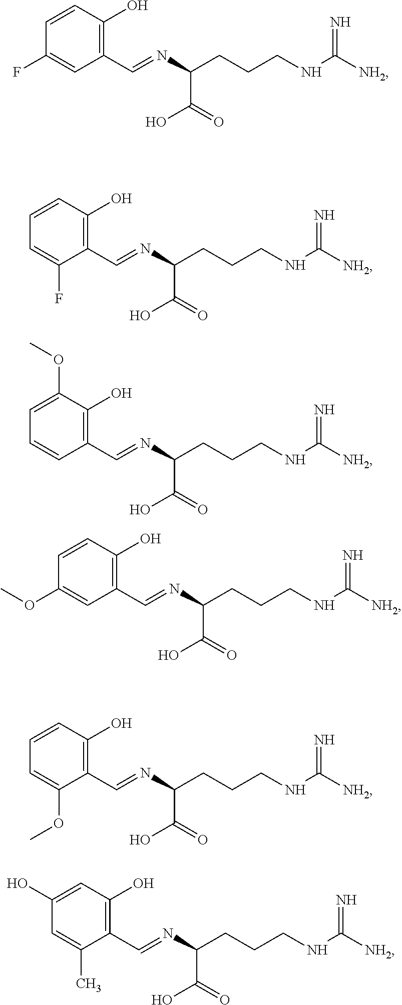 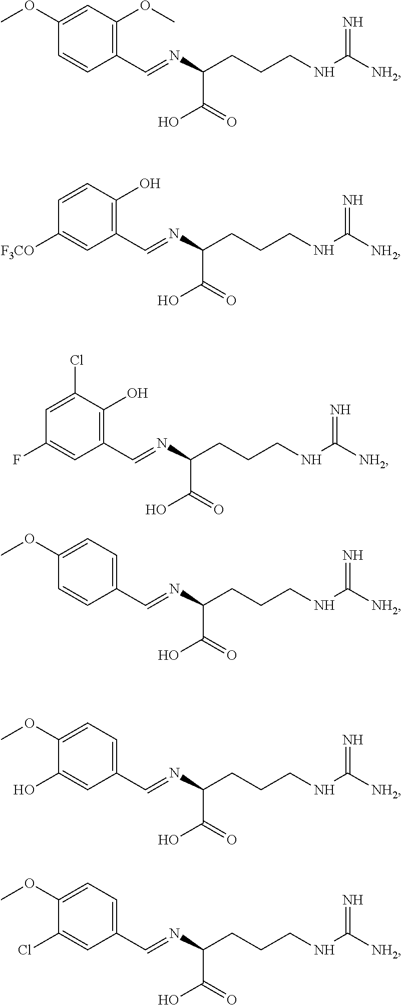 [SMILES: COc1ccc(/C=N/[C@@H](CCCNC(=N)N)C(=O)O)c(OC)c1 ; N=C(N)NCCC[C@H](/N=C/c1cc(OC(F)(F)F)ccc1O)C(=O)O ; N=C(N)NCCC[C@H](/N=C/c1cc(F)cc(Cl)c1O)C(=O)O ; COc1ccc(/C=N/[C@@H](CCCNC(=N)N)C(=O)O)cc1 ; COc1ccc(/C=N/[C@@H](CCCNC(=N)N)C(=O)O)cc1O ; COc1ccc(/C=N/[C@@H](CCCNC(=N)N)C(=O)O)cc1Cl] 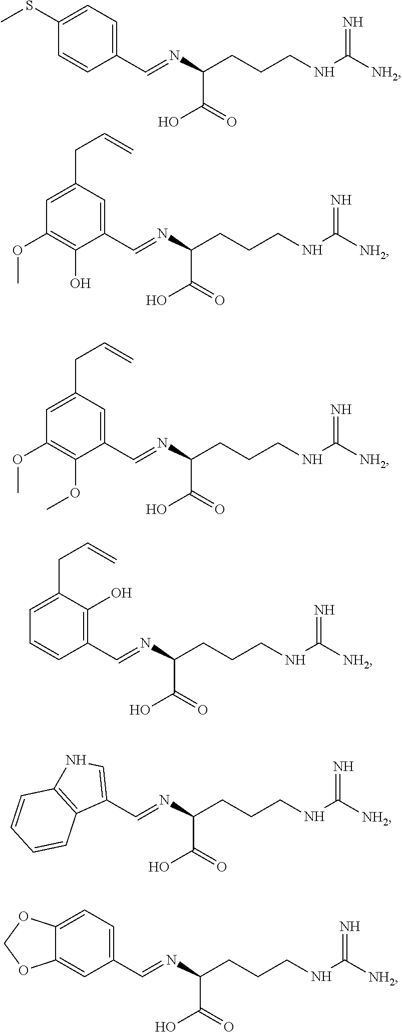 [SMILES: CSc1ccc(/C=N/[C@@H](CCCNC(=N)N)C(=O)O)cc1 ; C=CCc1cc(/C=N/[C@@H](CCCNC(=N)N)C(=O)O)c(O)c(OC)c1 ; C=CCc1cc(/C=N/[C@@H](CCCNC(=N)N)C(=O)O)c(OC)c(OC)c1 ; C=CCc1cccc(/C=N/[C@@H](CCCNC(=N)N)C(=O)O)c1O ; N=C(N)NCCC[C@H](/N=C/c1c[nH]c2ccccc12)C(=O)O ; N=C(N)NCCC[C@H](/N=C/c1ccc2c(c1)OCO2)C(=O)O] 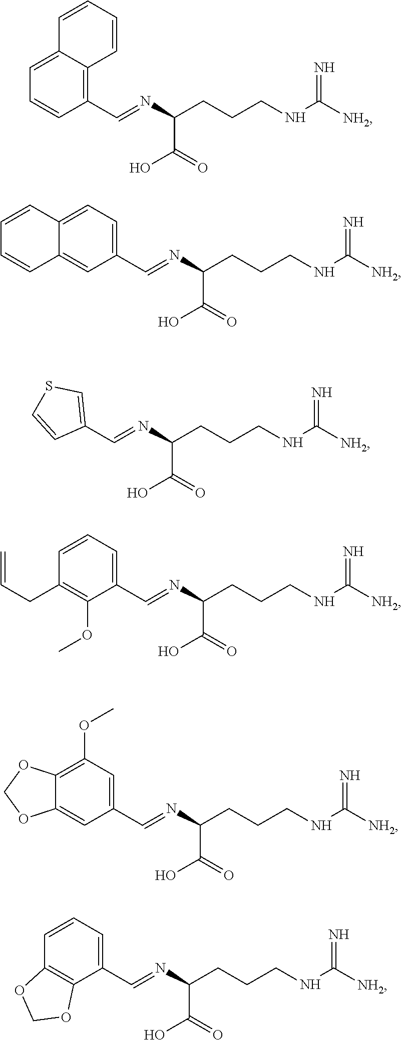 [SMILES: N=C(N)NCCC[C@H](/N=C/c1cccc2ccccc12)C(=O)O ; N=C(N)NCCC[C@H](/N=C/c1ccc2ccccc2c1)C(=O)O ; N=C(N)NCCC[C@H](/N=C/c1ccsc1)C(=O)O ; C=CCc1cccc(/C=N/[C@@H](CCCNC(=N)N)C(=O)O)c1OC ; COc1cc(/C=N/[C@@H](CCCNC(=N)N)C(=O)O)cc2c1OCO2 ; N=C(N)NCCC[C@H](/N=C/c1cccc2c1OCO2)C(=O)O]
or agrochemically acceptable salts or N-oxides thereof.

31. The agricultural composition according to claim 28, wherein the compound is selected from 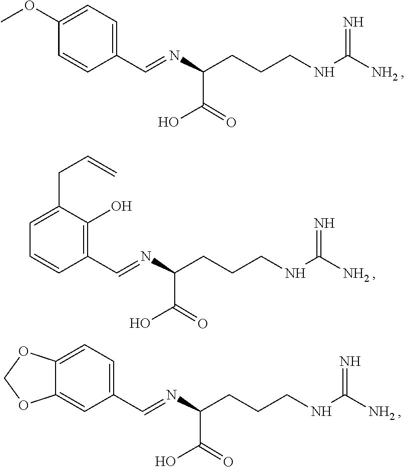 [SMILES: COc1ccc(/C=N/[C@@H](CCCNC(=N)N)C(=O)O)cc1 ; C=CCc1cccc(/C=N/[C@@H](CCCNC(=N)N)C(=O)O)c1O ; N=C(N)NCCC[C@H](/N=C/c1ccc2c(c1)OCO2)C(=O)O]
or agrochemically acceptable salts or N-oxides thereof.

32. The agricultural composition according to claim 28, wherein the compound is 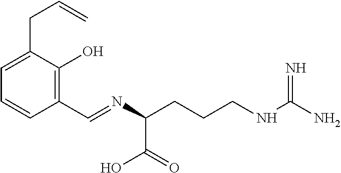 [SMILES: C=CCc1cccc(/C=N/[C@@H](CCCNC(=N)N)C(=O)O)c1O]
or an agrochemically acceptable salt or N-oxide thereof.

33. The agricultural composition according to claim 28, wherein the composition is in the form of a solid, a liquid, a gel, or a paste.

34. The agricultural composition according to claim 33, wherein the solid composition is in the form of spheronised granules, extruded granules, water disintegrable granules, wettable powders, water dispersible granules, dustable powder, powder for dry seed treatment, water disintegrable tablet, or water dispersible powder for slurry seed treatment.

35. The agricultural composition according to claim 33, wherein the liquid composition is in the form of liquid suspension, liquid suspension concentrate, suspoemulsion, oil dispersion, flowable concentrate, or a suspension concentrate for seed treatment.

36. A method of treating or controlling the parasitic nematode or the nematode infection in a plant comprising administering a compound of claim 23.

37. The method of claim 36, wherein the compound is selected from 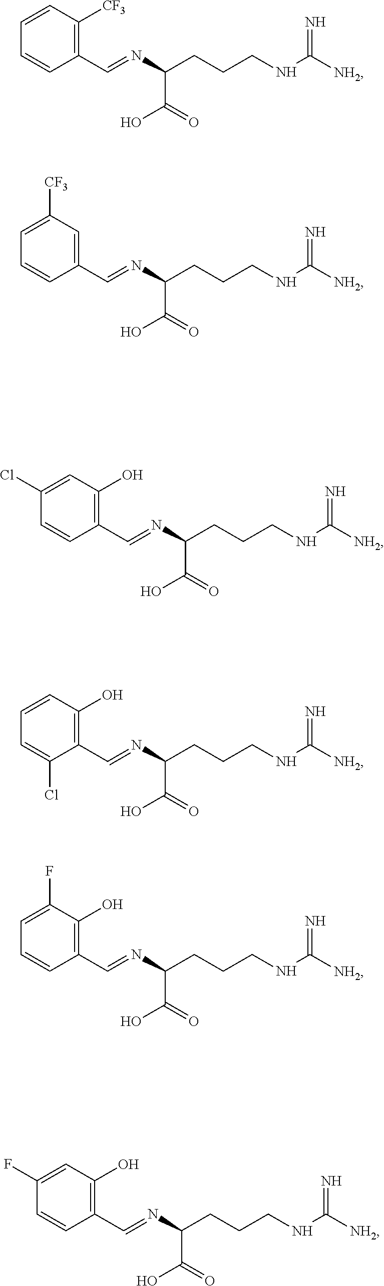 [SMILES: N=C(N)NCCC[C@H](/N=C/c1ccccc1C(F)(F)F)C(=O)O ; N=C(N)NCCC[C@H](/N=C/c1cccc(C(F)(F)F)c1)C(=O)O ; N=C(N)NCCC[C@H](/N=C/c1ccc(Cl)cc1O)C(=O)O ; N=C(N)NCCC[C@H](/N=C/c1c(O)cccc1Cl)C(=O)O ; N=C(N)NCCC[C@H](/N=C/c1cccc(F)c1O)C(=O)O ; N=C(N)NCCC[C@H](/N=C/c1ccc(F)cc1O)C(=O)O] 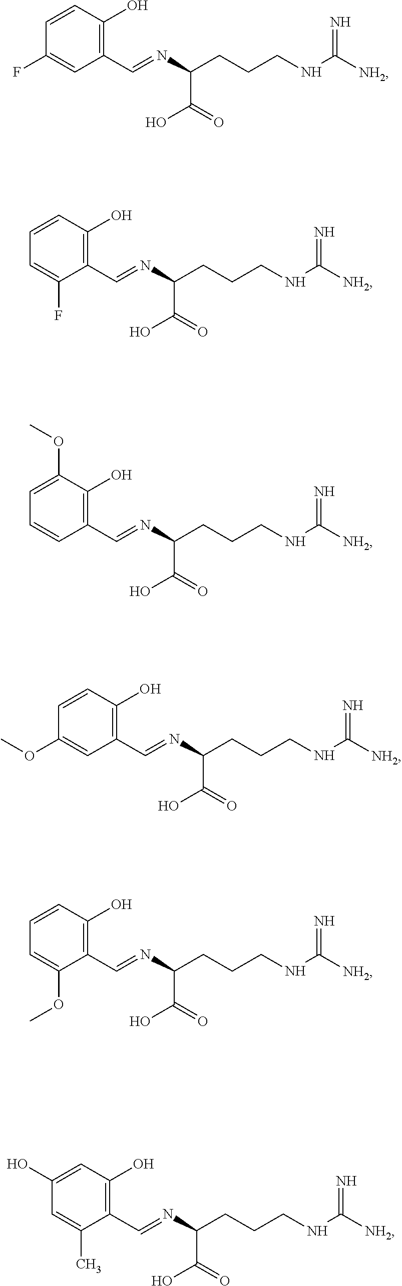 [SMILES: N=C(N)NCCC[C@H](/N=C/c1cc(F)ccc1O)C(=O)O ; N=C(N)NCCC[C@H](/N=C/c1c(O)cccc1F)C(=O)O ; COc1cccc(/C=N/[C@@H](CCCNC(=N)N)C(=O)O)c1O ; COc1ccc(O)c(/C=N/[C@@H](CCCNC(=N)N)C(=O)O)c1 ; COc1cccc(O)c1/C=N/[C@@H](CCCNC(=N)N)C(=O)O ; Cc1cc(O)cc(O)c1/C=N/[C@@H](CCCNC(=N)N)C(=O)O] 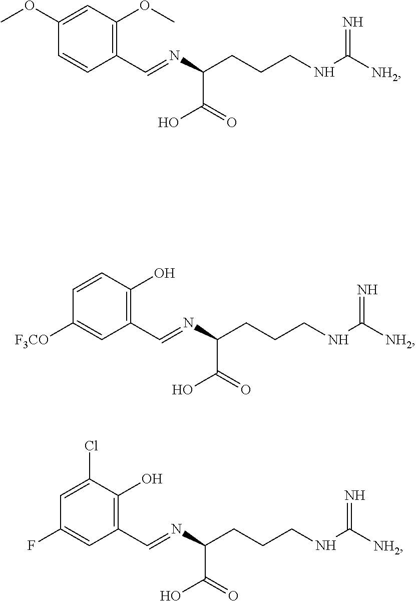 [SMILES: COc1ccc(/C=N/[C@@H](CCCNC(=N)N)C(=O)O)c(OC)c1 ; N=C(N)NCCC[C@H](/N=C/c1cc(OC(F)(F)F)ccc1O)C(=O)O ; N=C(N)NCCC[C@H](/N=C/c1cc(F)cc(Cl)c1O)C(=O)O] 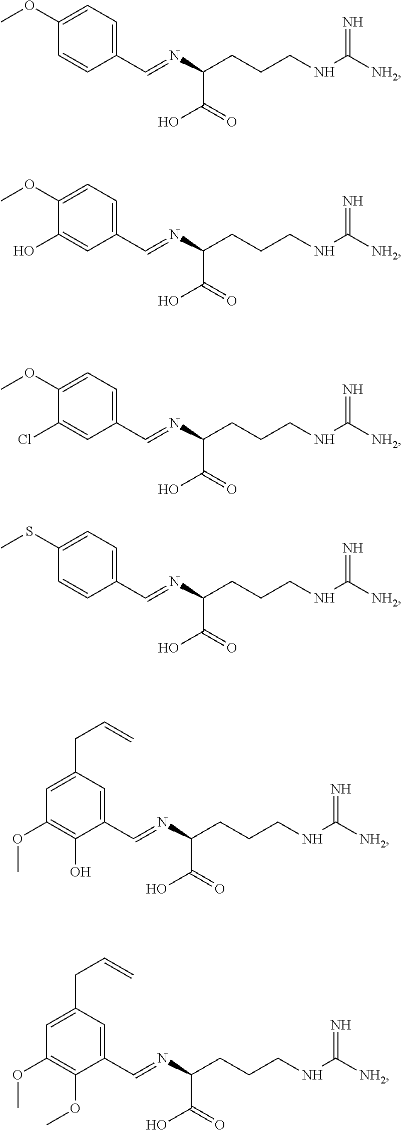 [SMILES: COc1ccc(/C=N/[C@@H](CCCNC(=N)N)C(=O)O)cc1 ; COc1ccc(/C=N/[C@@H](CCCNC(=N)N)C(=O)O)cc1O ; COc1ccc(/C=N/[C@@H](CCCNC(=N)N)C(=O)O)cc1Cl ; CSc1ccc(/C=N/[C@@H](CCCNC(=N)N)C(=O)O)cc1 ; C=CCc1cc(/C=N/[C@@H](CCCNC(=N)N)C(=O)O)c(O)c(OC)c1 ; C=CCc1cc(/C=N/[C@@H](CCCNC(=N)N)C(=O)O)c(OC)c(OC)c1] 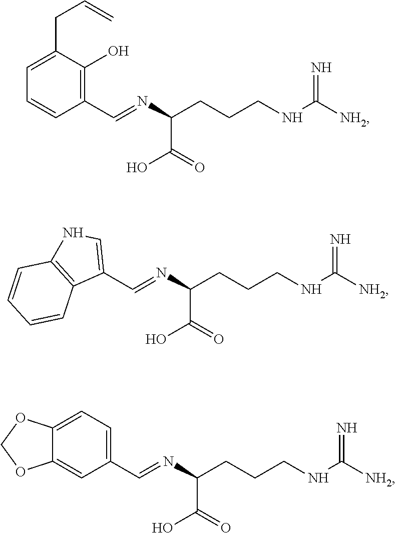 [SMILES: C=CCc1cccc(/C=N/[C@@H](CCCNC(=N)N)C(=O)O)c1O ; N=C(N)NCCC[C@H](/N=C/c1c[nH]c2ccccc12)C(=O)O ; N=C(N)NCCC[C@H](/N=C/c1ccc2c(c1)OCO2)C(=O)O] 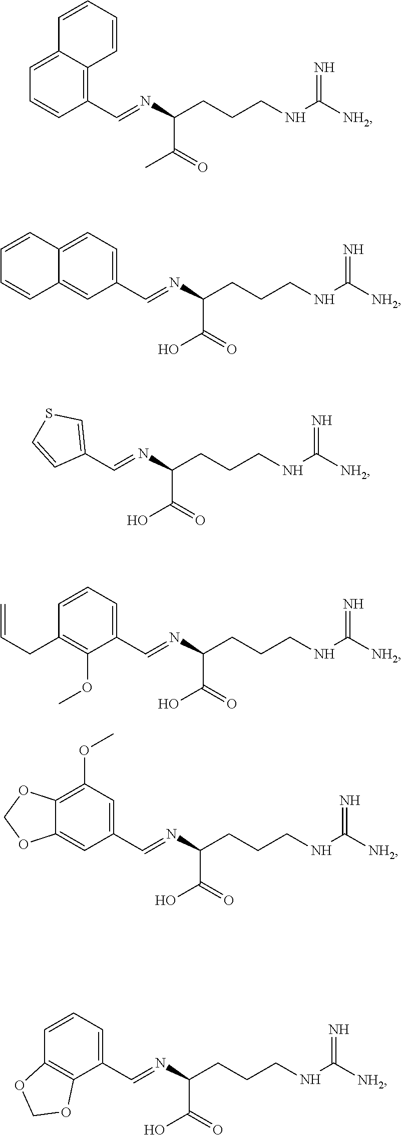 [SMILES: CC(=O)[C@H](CCCNC(=N)N)/N=C/c1cccc2ccccc12 ; N=C(N)NCCC[C@H](/N=C/c1ccc2ccccc2c1)C(=O)O ; N=C(N)NCCC[C@H](/N=C/c1ccsc1)C(=O)O ; C=CCc1cccc(/C=N/[C@@H](CCCNC(=N)N)C(=O)O)c1OC ; COc1cc(/C=N/[C@@H](CCCNC(=N)N)C(=O)O)cc2c1OCO2 ; N=C(N)NCCC[C@H](/N=C/c1cccc2c1OCO2)C(=O)O]
or agrochemically acceptable salts or N-oxides thereof.

38. The method according to claim 36, wherein the corn und is selected from 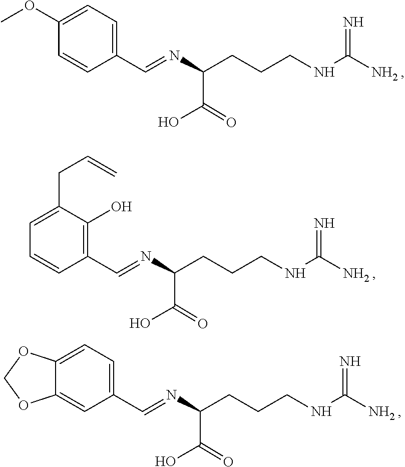 [SMILES: COc1ccc(/C=N/[C@@H](CCCNC(=N)N)C(=O)O)cc1 ; C=CCc1cccc(/C=N/[C@@H](CCCNC(=N)N)C(=O)O)c1O ; N=C(N)NCCC[C@H](/N=C/c1ccc2c(c1)OCO2)C(=O)O]
or agrochemically acceptable salts or N-oxides thereof.

39. A process for the preparation of compound of formula (I) which comprises combining a compound of formula (A) with a compound of Formula (B) 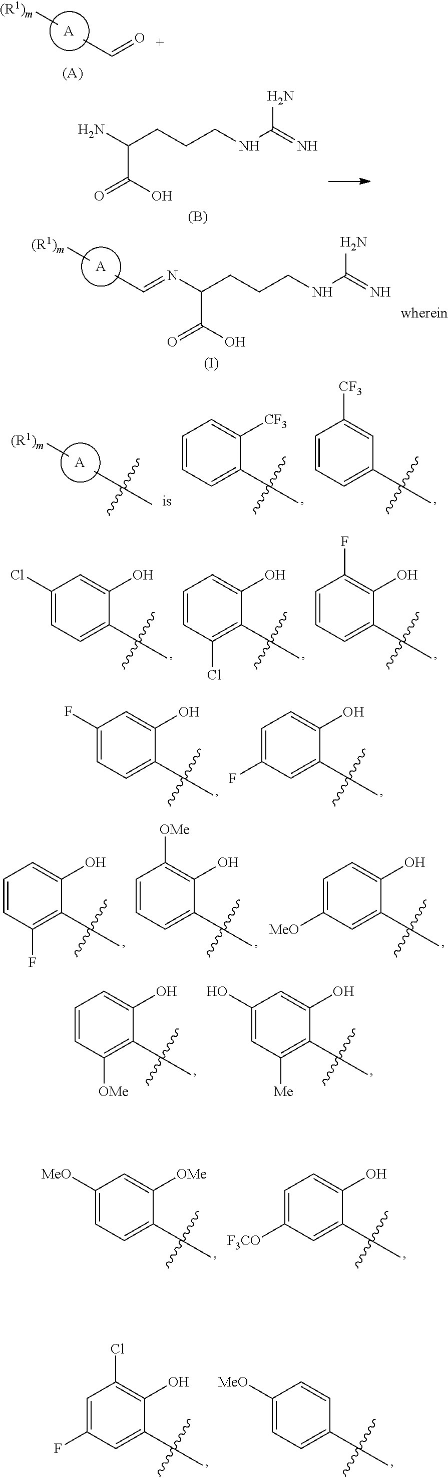 [chemical structure]
